(19) United States
(12) Patent Application Publication
(10) Pub. No.: US 2024/0009179 A1
(43) Pub. Date: Jan. 11, 2024

(54) METHODS OF TREATING CYSTIC FIBROSIS USING WITH-NO-LYSINE (WNK) KINASE PATHWAY INHIBITORS

(71) Applicant: UNIVERSITY OF IOWA RESEARCH FOUNDATION, Iowa City, IA (US)

(72) Inventors: Tayyab Rehman, Iowa City, IA (US); Michael J. Welsh, Iowa City, IA (US)

(21) Appl. No.: 18/347,192

(22) Filed: Jul. 5, 2023

Related U.S. Application Data
(60) Provisional application No. 63/358,411, filed on Jul. 5, 2022.

(51) Int. Cl. A61K 31/4545; A61P 11/00; G01N 33/50

(52) U.S. Cl. CPC A61K 31/4545; A61P 11/00; G01N 33/5044; G01N 33/5041

(57) ABSTRACT

Disclosed are methods for the treatment of cystic fibrosis, methods of increasing airway surface liquid pH, methods of enhancing respiratory defense, and methods of testing a compound of interest for use as a treatment for cystic fibrosis.

### CROSS-REFERENCE TO RELATED PATENT APPLICATIONS

The present application claims the benefit of priority to U.S. Provisional Patent Application 63/358,411, filed on Jul. 5, 2022. The content of the above-referenced application is incorporated herein by reference in its entirety.

This invention was made with government support under HL091842 and HL152960 awarded by the National Institutes of Health. The Government has certain rights in the invention.

### SEQUENCE LISTING

A Sequence Listing accompanies this application and is submitted as an XML file of the sequence listing named “139766_00111.xml” which is 21,269 bytes in size and was created on Jul. 5, 2023. The sequence listing is electronically submitted via Patent Center with the application and is incorporated herein by reference in its entirety.

### BACKGROUND

Cystic fibrosis (CF) is an inherited disorder caused by mutations in the CFTR gene. The protein encoded by this gene forms a pore in cells lining the airways and permits anions such as bicarbonate and chloride to be secreted. Due to loss of CFTR-mediated bicarbonate secretion, the airway surface liquid (ASL) becomes abnormally acidic, and acidic pH disrupts host defenses. Therefore, there is a need in the art for methods to increase airway pH in subjects suffering from cystic fibrosis.

### SUMMARY

Provided herein are methods of treating cystic fibrosis in a subject in need thereof, the methods comprising: administering to the subject an effective amount of a WNK kinase pathway inhibitor to treat the cystic fibrosis.

Also provided herein are methods of treating one or more signs or symptoms of cystic fibrosis in a subject in need thereof, the methods comprising: administering to the subject an effective amount of a WNK kinase pathway inhibitor to the subject to treat the one or more signs or symptoms of cystic fibrosis. In some embodiments, the one or more signs or symptoms of cystic fibrosis are selected from the group consisting of persistent cough, wheezing, exercise intolerance, and repeated lung infections.

Also provided herein are methods of increasing the pH of airway surface liquid (ASL) in a subject in need thereof, the methods comprising: administering to the subject an effective amount of a WNK kinase pathway inhibitor to increase the pH of the ASL in the subject.

Also provided herein are methods of enhancing respiratory defense in a subject in need thereof, the methods comprising: administering to the subject an effective amount of a WNK kinase pathway inhibitor to enhance respiratory defense in the subject.

In some embodiments of any of the above methods, administering the WNK kinase pathway inhibitor causes increased secretion of HCO₃⁻ ions in the airway surface liquid of the subject, and/or the subject has a mutation in at least one allele of cystic fibrosis transmembrane conductance regulator (CFTR) gene. In some embodiments, the mutation is F₅₀₈^{del}. In some embodiments, the mutation is not F₅₀₈^{del}. In some embodiments, the mutation is selected from a mutation, or combination of mutations, listed in Table 1.

In some embodiments of any of the disclosed methods, the WNK kinase pathway inhibitor is selected from a WNK1 inhibitor, a WNK2 inhibitor, a SPAK kinase inhibitor, or an OSR1 kinase inhibitor. In some embodiments, the WNK kinase pathway inhibitor inhibits WNK1 activity and/or WNK2 activity in airway epithelial cells in the subject. In some embodiments, the WNK kinase pathway inhibitor selectively inhibits WNK2. In some embodiments, the WNK kinase pathway inhibitor comprises a small molecule. In some embodiments, the WNK kinase pathway inhibitor comprises WNK463.

In some embodiments of the methods disclosed herein, the method reduces Na—K-2C1 cotransporter-1 (NKCC1) activity.

In some embodiments of the methods disclosed herein, the WNK kinase pathway inhibitor is administered intrapulmonarily.
18. Also provided herein are methods of testing a compound of interest for use as a treatment for cystic fibrosis, comprising: (a) culturing cells in the presence and absence of the compound of interest; (b) culturing control cells in the presence and absence of a WNK kinase pathway inhibitor; (c) detecting one or more parameters related to lung airway function in the cultured cells of step (a) and the cultured cells of step (b); (d) generating a test index by calculating the change in the one or more parameters between the cultured cells of step (a) in the presence and absence of the compound of interest; and (e) generating a control index by calculating the change in the one or more parameters between the cultured cells of step (b) in the presence and absence of the WNK kinase pathway inhibitor; wherein if the value of the test index is equal to, or improved, as compared to the value of the control index, then the compound of interest is of use for the treatment of cystic fibrosis. In some embodiments, the method further comprising treating cystic fibrosis in a subject in need thereof by administering to the subject an effective amount of the compound of interest to treat cystic fibrosis. In some embodiments, the cells comprise differentiated primary human airway epithelial cells. In some embodiments, the cells comprise NuLi-1 or CuFi-4 cells. In some embodiments, the cells have a mutation in at least one allele of cystic fibrosis transmembrane conductance regulator (CFTR) gene. In some embodiments, the mutation is F508^{del}. In some embodiments, the mutation is not F508^{del}. In some embodiments, the mutation is selected from a mutation or combination of mutations listed in Table 1. In some embodiments, the WNK kinase pathway inhibitor is selected from a WNK1 inhibitor, a WNK2 inhibitor, a SPAK kinase inhibitor, or an OSR1 kinase inhibitor. In some embodiments, the WNK kinase pathway inhibitor comprises a small molecule. In some embodiments, the WNK kinase pathway inhibitor comprises WNK463. In some embodiments, the WNK kinase pathway inhibitor selectively inhibits WNK2. In some embodiments, the WNK kinase pathway inhibitor reduces Na—K-2Cl cotransporter-1 (NKCC1) activity. In some embodiments, the one or more parameters are selected from pH, HCO₃⁻ secretion, Cl⁻ secretion, mucus viscosity, bacterial colonization, and electrical conductance across the membrane of the cells.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-ID. Expression of WNK kinases in human airway epithelia. FIG. 1A) Single-cell RNA-seq was performed on primary cultures of differentiated airway epithelia from non-CF and CF donors (n=4 per genotype). Dot plot showing cell type-specific expression of the four WNK isoforms, and STK39 and OXSR1 which encode SPAK and OSR1, respectively. For each dot, the size represents the detection rate in a particular cell type, and the color represent average gene expression for cells in which gene was detected. FIGS. 1B-1D) Confocal images showing WNK1 and WNK2 immunolocalization in CF epithelia. Scale bar: 5 μm. Similar staining results were obtained in 2 different donors.

FIGS. 2A-2H. WNK463 increases pH_{ASL}. Human airway epithelia were exposed to either vehicle or WNK463 (10 μM) for 2 hours, and pH_{ASL} measured using SNARF-1-dextran. FIG. 2A and FIG. 2B) pH_{ASL} responses in primary cultures of non-CF (n=7) and CF epithelia (n=8). FIG. 2C and FIG. 2D) pH_{ASL} responses in NuLi-1 (n=6) and CuFi-4 epithelia (n=6). FIG. 2E) pH_{ASL} response in primary CF epithelia in the presence of HCO₃⁻/CO₂, and after replacing HCO₃⁻ with HEPES and removing CO₂ from the environment (n=4). FIG. 2F) Time course of WNK463⁻evoked response in primary CF epithelia (n=5). FIG. 2G) Schematic showing direct versus indirect modulation of membrane transporters by WNK kinases. FIG. 2H) pH_{ASL} response in primary CF epithelia after 2-hour exposure to rafoxanide, a SPAK/OSR1 inhibitor (n=4). In FIG. 2C and FIG. 2D, each data point is a technical replicate. In all other cases, each data point represents an epithelium from a different human donor. Data are shown as mean±SEM. Statistical significance was tested using paired Student's t test for FIGS. 2A-2D, and ANOVA with post-test Tukey's for FIGS. 2E, 2F, and 2H. *P<0.05, **P<0.01, ***P<0.001.

FIGS. 3A-3D. WNK463 enhances CF host defenses. Primary cultures of differentiated CF airway epithelia were treated with either vehicle or WNK463 (10 μM). All treatments were for 2 hours except B where it was for 4 hours. FIG. 3A) ASL viscosity (τ_{ASL}/τₛₐₗᵢₙₑ) in primary CF epithelia (n=5). The dashed horizontal line indicates the viscosity of saline. FIG. 3B) Rate of apical liquid absorption in primary CF epithelia (n=6). The dashed horizontal line at 0 indicates no net secretion or absorption. FIG. 3C) Ciliary beat frequency (CBF) in primary CF epithelia (n=11). FIG. 3D) ASL killing activity against S. aureus in primary CF epithelia (n=5). Each data point represents an epithelium from a different donor. Data are shown as mean±SEM. Statistical significance was tested using paired Student's t test. *P<0.05, **P<0.01.

FIGS. 4A-4D. WNK1 and WNK2 regulate CF pH_{ASL}. siRNAs were used to knockdown gene expression in primary CF epithelia, and pH_{ASL} measured using SNARF-1-dextran. Knockdown efficiency was assessed using qRT-PCR. FIG. 4A and FIG. 4B) knockdown of WNK1 (n=5). FIG. 4C and FIG. 4D) knockdown of WNK2 (n=6). Each data point represents an epithelium from a different donor. Statistical significance was tested using paired Student's t test. *P<0.05, **P<0.01. NC, negative control.

FIGS. 5A-5J. Effect of WNK463 on electrogenic Cl⁻ and HCO₃⁻ secretion. Primary cultures of differentiated airway epithelia were treated with either vehicle (DMSO) or WNK463 (10 μM) for 2 hours. Epithelia were mounted in Ussing chambers and I_{SC} and Gₜ were recorded as agents were sequentially added to the apical side. FIG. 5A and FIG. 5B) I_{SC} and Gₜ in non-CF epithelia (n=5). FIG. 5C and FIG. 5D) ΔI_{SC} and ΔGₜ with addition of CFTRᵢₙₕ-172 in non-CF epithelia (n=5). FIG. 5E and FIG. 5F) I_{SC} and Gₜ in CF epithelia (n=7). FIG. 5G and FIG. 5H) ΔI_{SC} and ΔGₜ with addition of apical DIDS in CF epithelia (n=7). Studies in FIGS. 5A-5H were performed with symmetric buffers containing both Cl⁻ and HCO₃⁻. FIG. 5I and FIG. 5J) To separate the effect of WNK463 on electrogenic Cl⁻ versus HCO₃⁻ transport, Ussing chamber studies were repeated with HCO₃⁻-free or Cl⁻-free solutions. (FIG. 5I) shows ΔI_{SC} response with addition of CFTRᵢₙₕ-172 in non-CF epithelia (n=4-7), and (FIG. 5J) shows ΔI_{SC} response with apical DIDS in CF epithelia (n=5-6). Each data point represents an epithelium from a different donor. Data are shown as mean SEM. In some cases, error bars are hidden by symbols. Statistical significance was tested using paired Student's t test. *P<0.05.

FIGS. 6A-6D. Reducing C1⁻ transport increases CF pH_{ASL}. FIG. 6A and FIG. 6B) Primary cultures of differentiated airway epithelia were treated with either vehicle or WNK463 (10 μM) for 2 hours, and assayed in Ussing chambers containing symmetric Krebs buffer containing both Cl⁻ and HCO₃⁻. During I_{SC} recording, amiloride was added to inhibit ENaC, followed by forskolin (non-CF) or UTP (CF) to maximally activate CFTR or CaCC, respectively. At this point, basolateral bumetanide was introduced, and ΔI_{SC} was recorded (n=6 different non-CF, or 9 different CF donors). FIG. 6C) Left panel: CF pH_{ASL} response 2 hours after exposure to basolateral bumetanide (n=6 different donors); right panel: CF pH_{ASL} in the presence or absence of basolateral Cl⁻ (n=6 different donors). FIG. 6D) CF pH_{ASL} response in epithelia exposed to WNK463 (10 μM) for 2 hours in the presence or absence of basolateral Cl⁻(n=6 different donors). Data are shown as mean±SEM. Statistical significance was tested using paired Student's t test. *P<0.05, **P<0.01.

FIGS. 7A-7E. pH_{ASL} response to WNK463 in TNFα/IL-17⁻treated CF epithelia. Primary cultures of differentiated CF epithelia were treated with TNFα (10 ng/ml) and IL-17 (20 ng/ml) for 48 hours. FIG. 7A) Changes in WNK gene expression revealed by bulk RNA-seq (n=6). TPM, transcripts per million. FIG. 7B) WNK1 and WNK2 mRNA expression measured using qRT-PCR (n=5). FIG. 7C) Immunostaining for WNK1 and WNK2 in control and TNFα/IL-17⁻treated CF epithelia. Scale bar: 5 μm. FIG. 7D) Intensity of WNK1 or WNK2 immunolabeling quantitated as integrated density using the imageJ software (n=5-6). FIG. 7E and FIG. 7F) pH_{ASL} responses in TNFα/IL-17⁻treated CF epithelia. WNK463 (10 μM) or bumetanide (100 μM) were applied for 2 hours prior to pH_{ASL} measurement (n=6-8). Each data point represents an epithelium from a different donor. Data are shown as mean±SEM. Statistical significance was tested using paired Student's t test (FIG. 7A, FIG. 7B, FIG. 7D and FIG. 7F) or ANOVA with post-test Tukey's (FIG. 7E). *P<0.05, **P<0.01, ****P<0.0001.

FIGS. 8A-8F. Expression of genes encoding WNK/SPAK/OSR1 kinases in CF versus non-CF epithelia. Single-cell RNA-seq was performed on primary cultures of differentiated human airway epithelia from non-CF (blue) and CF (red) donors. N=4 per genotype. FIGS. 8A-8D) Cell type-specific expression of WNK isoforms. FIG. 8E and FIG. 8F) Cell type-specific expression of STK39 and OXSR1, which encode SPAK and OSR1 respectively. Each data point represents an epithelium from a different donor. Bars indicate mean. Data for CF ionocytes is not shown as these cells were not detected in 3 out of 4 CF epithelia.

### DETAILED DESCRIPTION

The present invention is described herein using several definitions, as set forth below and throughout the application.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

Methods of Treatment

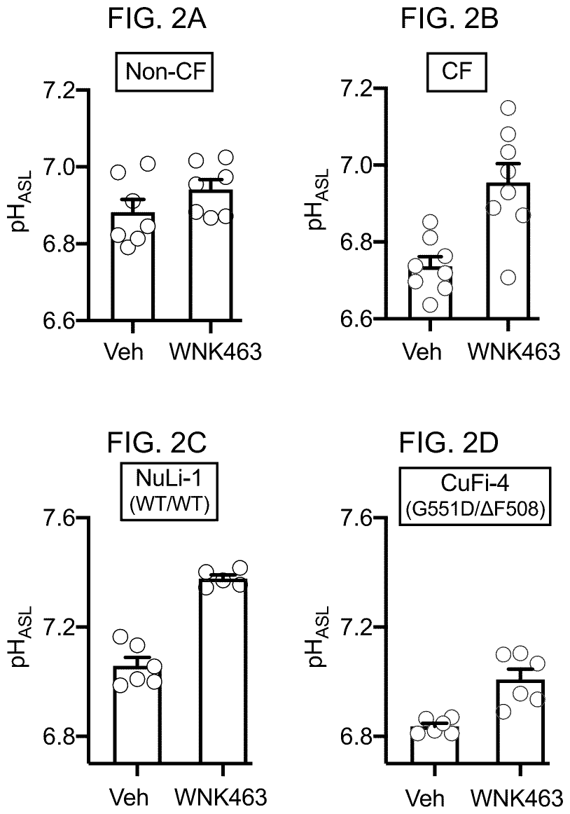

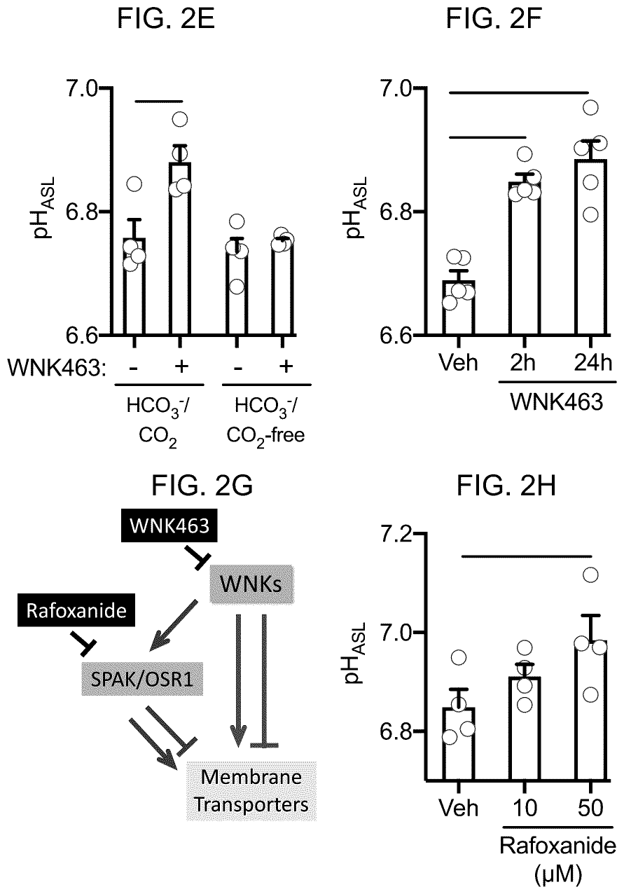

The inventors discovered that with-no-lysine (WNK) kinases regulate the pH of airway surface liquid (ASL). Furthermore, the inventors discovered that inhibiting WNK kinases, or the downstream kinases SPAK or OSR1, increases the pH of ASL by increasing the secretion of HCO₃⁻ ions into the ASL (FIGS. 2A and B). The pH of ASL is critically important to the health of the respiratory tract as the relatively high pH of the lower airway, about 7, is required for normal host defense. As discussed above, mutations in the gene cystic fibrosis transmembrane conductance regulator (CFTR) causes cystic fibrosis in affected individuals—which is associated with a lower pH of the ASL. The inventors discovered that inhibition of WNK, SPAK, or OSR1 kinases increases the pH of ASL and improves host defenses in models of cystic fibrosis.

Accordingly, in a first aspect of the current disclosure, methods of treating cystic fibrosis in a subject in need thereof are provided. In some embodiments, the methods comprise: administering to the subject an effective amount of a WNK kinase pathway inhibitor to treat the cystic fibrosis.

As used herein, “a subject in need thereof” refers to a subject suffering from cystic fibrosis or a subject in need of increased airway surface liquid pH. The term “subject” may be used interchangeably with the terms “individual” and “patient” and includes human and non-human mammalian subjects.

As briefly discussed above, two copies of mutations in CFTR causes cystic fibrosis. Thus, mutant CFTR alleles are considered to be recessive alleles. A variety of mutations in CFTR have been described, including those listed below in Table 1. Therefore, a subject in need thereof, in some embodiments, comprises a subject with a mutation in CFTR selected from those listed in Table 1.

| Human CFTR has the amino acid sequence (SEQ ID NO: 1): | |
|---|---|
| MQRSPLEKAS VVSKLFFSWT RPILRKGYRQ RLELSDIYQI PSVDSADNLS EKLEREWDRE | 60 |
| LASKKNPKLI NALRRCFFWR FMFYGIFLYL GEVTKAVQPL LLGRIIASYD PDNKEERSIA | 120 |
| IYLGIGLCLL FIVRTLLLHP AIFGLHHIGM QMRIAMFSLI YKKTLKLSSR VLDKISIGQL | 180 |
| VSLLSNNLNK FDEGLALAHF VWIAPLQVAL LMGLIWELLQ ASAFCGLGFL IVLALFQAGL | 240 |
| GRMMMKYRDQ RAGKISERLV ITSEMIENIQ SVKAYCWEEA MEKMIENLRQ TELKLTRKAA | 300 |
| YVRYFNSSAF FFSGFFVVFL SVLPYALIKG IILRKIFTTI SFCIVLRMAV TRQFPWAVQT | 360 |
| WYDSLGAINK IQDFLQKQEY KTLEYNLTTT EVVMENVTAF WEEGFGELFE KAKQNNNNRK | 420 |
| TSNGDDSLFF SNFSLLGTPV LKDINFKIER GQLLAVAGST GAGKTSLLMV IMGELEPSEG | 480 |
| KIKHSGRISF CSQFSWIMPG TIKENIIFGV SYDEYRYRSV IKACQLEEDI SKFAEKDNIV | 540 |
| LGEGGITLSG GQRARISLAR AVYKDADLYL LDSPFGYLDV LTEKEIFESC VCKLMANKTR | 600 |
| ILVTSKMEHL KKADKILILH EGSSYFYGTF SELQNLQPDF SSKLMGCDSF DQFSAERRNS | 660 |
| ILTETLHRES LEGDAPVSWT ETKKQSFKQT GEFGEKRKNS ILNPINSIRK FSIVQKTPLQ | 720 |
| MNGIEEDSDE PLERRLSLVP DSEQGEAILP RISVISTGPT LQARRRQSVL NLMTHSVNQG | 780 |
| QNIHRKTTAS TRKVSLAPQA NLTELDIYSR RLSQETGLEI SEEINEEDLK ECFFDDMESI | 840 |
| PAVTTWNTYL RYITVHKSLI FVLIWCLVIF LAEVAASLVV LWLLGNTPLQ DKGNSTHSRN | 900 |
| NSYAVIITST SSYYVFYIYV GVADTLLAMG FFRGLPLVHT LITVSKILHH KMLHSVLQAP | 960 |
| MSTLNTLKAG GILNRFSKDI AILDDLLPLT IFDFIQLLLI VIGAIAVVAV LQPYIFVATV | 1020 |
| PVIVAFIMLR AYFLQTSQQL KQLESEGRSP IFTHLVTSLK GLWTLRAFGR QPYFETLFHK | 1080 |
| ALNLHTANWF LYLSTLRWFQ MRIEMIFVIF FIAVTFISIL TTGEGEGRVG IILTLAMNIM | 1140 |
| STLQWAVNSS IDVDSLMRSV SRVFKFIDMP TEGKPTKSTK PYKNGQLSKV MIIENSHVKK | 1200 |
| DDIWPSGGQM TVKDLTAKYT EGGNAILENI SFSISPGQRV GLLGRTGSGK STLLSAFLRL | 1260 |
| LNTEGEIQID GVSWDSITLQ QWRKAFGVIP QKVFIFSGTF RKNLDPYEQW SDQEIWKVAD | 1320 |
| EVGLRSVIEQ FPGKLDFVLV DGGCVLSHGH KQLMCLARSV LSKAKILLLD EPSAHLDPVT | 1380 |
| YQIIRRTLKQ AFADCTVILC EHRIEAMLEC QQFLVIEENK VRQYDSIQKL LNERSLERQA | 1440 |
| ISPSDRVKLF PHRNSSKCKS KPQIAALKEE TEEEVQDTRL | 1480 |

TABLE 1
Exemplary list of CFTR mutations. Mutations listed
are relative to wild type CFTR (SEQ ID NO: 1).

| | | | | | |
|---|---|---|---|---|---|
| 3141del9 | E822K | G1069R | L967S | R117L | S912L |
| 546insCTA | F191V | G1244E | L997F | R117P | S945L |
| A46D | F311del | G1249R | L1077P | R170H | S977F |
| A120T | F311L | G1349D | L1324P | R258G | S1159F |
| A234D | F508C | H139R | L1335P | R334L | S1159P |
| A349V | F508C; S1251N^{†} | H199Y | L1480P | R334Q | S1251N |
| A455E | F508del* | H939R | M152V | R347H | S1255P |
| A554E | F575Y | H1054D | M265R | R347L | T338I |
| A1006E | F1016S | H1085P | M952I | R347P | T1036N |
| A1067T | F1052V | H1085R | M952T | R352Q | T1053I |
| D110E | F1074L | H1375P | M1101K | R352W | V201M |
| D110H | F1099L | I148T | P5L | R553Q | V232D |
| D192G | G27R | I175V | P67L | R668C | V456A |
| D443Y | G85E | I336K | P205S | R751L | V456F |
| D443Y; G576A; | G126D | I502T | P574H | R792G | V562I |
| R668C^{†} | | | | | |
| D579G | G178E | I601F | Q98R | R933G | V754M |
| D614G | G178R | I618T | Q237E | R1066H | V1153E |
| D836Y | G194R | I807M | Q237H | R1070Q | V1240G |
| D924N | G194V | 1980K | Q359R | R1070W | V1293G |
| D979V | G314E | I1027T | Q1291R | R1162L | W361R |
| D1152H | G463V | I1139V | R31L | R1283M | W1098C |
| D1270N | G480C | I1269N | R74Q | R1283S | W1282R |
| E56K | G551D | I1366N | R74W | S13F | Y109N |
| E60K | G551S | K1060T | R74W; D1270N^{†} | S341P | Y161D |
| E92K | G576A | L15P | R74W; V201M^{†} | S364P | Y161S |
| E116K | G576A; R668C^{†} | L165S | R74W; V201M; | S492F | Y563N |
| | | | D1270N^{†} | | |
| E193K | G622D | L206W | R75Q | S549N | Y1014C |
| E403D | G628R | L320V | R117C | S549R | Y1032C |
| E474K | G970D | L346P | R117G | S589N | |
| E588V | G1061R | L453S | R117H | S737F | |

The mutant alleles described in Table 1 are intended to be exemplary. Subjects With mutations in the CFTR gene that disrupt the normal function of CFTR are contemplated to be subjects in need thereof.

The WNK kinase family comprises four members: WNK1, WNK2, WNK3, and WNK4. The amino acid sequences for each of the human WNK kinases are listed below:

| Human WNK1 has the amino acid sequence (SEQ ID NO: 2): | |
|---|---|
| MLASPATETT VLMSQTEADL ALRPPPPLGT AGQPRLGPPP RRARRFSGKA EPRPRSSRLS | 60 |
| RRSSVDLGLL SSWSLPASPA PDPPDPPDSA GPGPARSPPP SSKEPPEGTW TEGAPVKAAE | 120 |
| DSARPELPDS AVGPGSREPL RVPEAVALER RREQEEKEDM ETQAVATSPD GRYLKFDIEI | 180 |
| GRGSFKTVYR GLDTDTTVEV AWCELQTRKL SRAERQRFSE EVEMLKGLQH PNIVRFYDSW | 240 |
| KSVLRGQVCI VLVTELMTSG TLKTYLRRFR EMKPRVLQRW SRQILRGLHF LHSRVPPILH | 300 |
| RDLKCDNVFI TGPTGSVKIG DLGLATLKRA SFAKSVIGTP EFMAPEMYEE KYDEAVDVYA | 360 |
| FGMCMLEMAT SEYPYSECQN AAQIYRKVTS GRKPNSFHKV KIPEVKEIIE GCIRTDKNER | 420 |
| FTIQDLLAHA FFREERGVHV ELAEEDDGEK PGLKLWLRME DARRGGRPRD NQAIEFLFQL | 480 |
| GRDAAEEVAQ EMVALGLVCE ADYQPVARAV RERVAAIQRK REKLRKAREL EALPPEPGPP | 540 |
| PATVPMAPGP PSVFPPEPEE PEADQHQPFL FRHASYSSTT SDCETDGYLS SSGFLDASDP | 600 |
| ALQPPGGVPS SLAESHLCLP SAFALSIPRS GPGSDFSPGD SYASDAASGL SDVGEGMGQM | 660 |
| RRPPGRNLRR RPRSRLRVTS VSDQNDRVVE CQLQTHNSKM VTFRFDLDGD SPEEIAAAMV | 720 |
| YNEFILPSER DGFLRRIREI IQRVETLLKR DTGPMEAAED TLSPQEEPAP LPALPVPLPD | 780 |
| PSNEELQSST SLEHRSWTAF STSSSSPGTP LSPGNPFSPG TPISPGPIFP ITSPPCHPSP | 840 |
| SPFSPISSQV SSNPSPHPTS SPLPFSSSTP EFPVPLSQCP WSSLPTTSPP TFSPTCSQVT | 900 |
| LSSPFFPPCP STSSFPSTTA APLLSLASAF SLAVMTVAQS LLSPSPGLLS QSPPAPPSPL | 960 |
| PSLPLPPPVA PGGQESPSPH TAEVESEASP PPARPLPGEA RLAPISEEGK PQLVGRFQVT | 1020 |
| SSKEPAEPLP LQPTSPTLSG SPKPSTPQLT SESSDTEDSA GGGPETREAL AESDRAAEGL | 1080 |
| GAGVEEEGDD GKEPQVGGSP QPLSHPSPVW MNYSYSSLCL SSEESESSGE DEEFWAELQS | 1140 |
| LRQKHLSEVE TLQTLQKKEI EDLYSRLGKQ PPPGIVAPAA MLSSRQRRLS KGSFPTSRRN | 1200 |
| SLQRSEPPGP GIMRRNSLSG SSTGSQEQRA SKGVTFAGDV GRM | 1243 |
| Human WNK2 has the amino acid sequence (SEQ ID NO: 3): | |
| MDGDGGRRDV PGTLMEPGRG AGPAGMAEPR AKAARPGPQR FLRRSVVESD QEEPPGLEAA | 60 |
| EAPGPQPPQP LQRRVLLLCK TRRLIAERAR GRPAAPAPAA LVAQPGAPGA PADAGPEPVG | 120 |
| TQEPGPDPIA AAVETAPAPD GGPREEAAAT VRKEDEGAAE AKPEPGRTRR DEPEEEEDDE | 180 |
| DDLKAVATSL DGRFLKFDIE LGRGSFKTVY KGLDTETWVE VAWCELQDRK LTKLERQRFK | 240 |
| EEAEMLKGLQ HPNIVRFYDF WESSAKGKRC IVLVTELMTS GTLKTYLKRF KVMKPKVLRS | 300 |
| WCRQILKGLL FLHTRTPPII HRDLKCDNIF ITGPTGSVKI GDLGLATLKR ASFAKSVIGT | 360 |
| PEFMAPEMYE EHYDESVDVY AFGMCMLEMA TSEYPYSECQ NAAQIYRKVT CGIKPASFEK | 420 |
| VHDPEIKEII GECICKNKEE RYEIKDLLSH AFFAEDTGVR VELAEEDHGR KSTIALRLWV | 480 |
| EDPKKLKGKP KDNGAIEFTF DLEKETPDEV AQEMIESGFF HESDVKIVAK SIRDRVALIQ | 540 |
| WRRERIWPAL QPKEQQDVGS PDKARGPPVP LQVQVTYHAQ AGQPGPPEPE EPEADQHLLP | 600 |
| PTLPTSATSL ASDSTFDSGQ GSTVYSDSQS SQQSVMLGSL ADAAPSPAQC VCSPPVSEGP | 660 |
| VLPQSLPSLG AYQQPTAAPG LPVGSVPAPA CPPSLQQHFP DPAMSFAPVL PPPSTPMPTG | 720 |
| PGQPAPPGQQ PPPLAQPTPL PQVLAPQPVV PLQPVPPHLP PYLAPASQVG APAQLKPLQM | 780 |
| PQAPLQPLAQ VPPQMPPIPV VPPITPLAGI DGLPPALPDL PTATVPPVPP PQYFSPAVIL | 840 |
| PSLAAPLPPA SPALPLQAVK LPHPPGAPLA MPCRTIVPNA PATIPLLAVA PPGVAALSIH | 900 |
| SAVAQLPGQP VYPAAFPQMA PTDVPPSPHH TVQNMRATPP QPALPPQPTL PPQPVLPPQP | 960 |
| TLPPQPVLPP QPTRPPQPVL PPQPMLPPQP VLPPQPALPV RPEPLQPHLP EQAAPAATPG | 1020 |
| SQILLGHPAP YAVDVAAQVP TVPVPPAAVL SPPLPEVLLP AAPELLPQFP SSLATVSASV | 1080 |
| QSVPTQTATL LPPANPPLPG GPGIASPCPT VQLTVEPVQE EQASQDKPPG LPQSCESYGG | 1140 |
| SDVTSGKELS DSCEGAFGGG RLEGRAARKH HRRSTRARSR QERASRPRLT ILNVCNTGDK | 1200 |
| MVECQLETHN HKMVTFKFDL DGDAPDEIAT YMVEHDFILQ AERETFIEQM KDVMDKAEDM | 1260 |
| LSEDTDADRG SDPGTSPPHL STCGLGTGEE SRQSQANAPV YQQNVLHTGK RWFIICPVAE | 1320 |
| HPAPEAPESS PPLPLSSLPP EASQGPCRGL TLPCLPWRRA ACGAVFLSLF SAESAQSKQP | 1380 |
| PDSAPYKDQL SSKEQPSFLA SQQLLSQAGP SNPPGAPPAP LAPSSPPVTA LPQDGAAPAT | 1440 |
| STMPEPASGT ASQAGGPGTP QGLTSELETS QPLAETHEAP LAVQPLVVGL APCTPAPEAA | 1500 |
| STRDASAPRE PLPPPAPEPS PHSGTPQPAL GQPAPLLPAA VGAVSLATSQ LPSPPLGPTV | 1560 |
| PPQPPSALES DGEGPPPRVG FVDSTIKSLD EKLRTLLYQE HVPTSSASAG TPVEVGDRDF | 1620 |
| TLEPLRGDQP RSEVCGGDLA LPPVPKEAVS GRVQLPQPLV EKSELAPTRG AVMEQGTSSS | 1680 |
| MTAESSPRSM LGYDRDGRQV ASDSHVVPSV PQDVPAFVRP ARVEPTDRDG GEAGESSAEP | 1740 |
| PPSDMGTVGG QASHPQTLGA RALGSPRKRP EQQDVSSPAK TVGRFSVVST QDEWTLASPH | 1800 |
| SLRYSAPPDV YLDEAPSSPD VKLAVRRAQT ASSIEVGVGE PVSSDSGDEG PRARPPVQKQ | 1860 |
| ASLPVSGSVA GDFVKKATAF LQRPSRAGSL GPETPSRVGM KVPTISVTSF HSQSSYISSD | 1920 |
| NDSELEDADI KKELQSLREK HLKEISELQS QQKQEIEALY RRLGKPLPPN VGFFHTAPPT | 1980 |
| GRRRKTSKSK LKAGKLLNPL VRQLKVVASS TGHLADSSRG PPAKDPAQAS VGLTADSTGL | 2040 |
| SGKAVQTQQP CSVRASLSSD ICSGLASDGG GARGQGWTVY HPTSERVTYK SSSKPRARFL | 2100 |
| SGPVSVSIWS ALKRLCLGKE HSSRSSTSSL APGPEPGPQP ALHVQAQVNN SNNKKGTFTD | 2160 |
| DLHKLVDEWT SKTVGAAQLK PTLNQLKQTQ KLQDMEAQAG WAAPGEARAM TAPRAGVGMP | 2220 |
| RLPPAPGPLS TTVIPGAAPT LSVPTPDGAL GTARRNQVWF GLRVPPTACC GHSTQPRGGQ | 2280 |
| RVGSKTASFA ASDPVRS | 2297 |
| Human WNK3 has the amino acid sequence (SEQ ID NO: 4): | |
| MATDSGDPAS TEDSEKPDGI SFENRVPQVA ATLTVEARLK EKNSTFSASG ETVERKRFFR | 60 |
| KSVEMTEDDK VAESSPKDER IKAAMNIPRV DKLPSNVLRG GQEVKYEQCS KSTSEISKDC | 120 |
| FKEKNEKEME EEAEMKAVAT SPSGRFLKFD IELGRGAFKT VYKGLDTETW VEVAWCELQD | 180 |
| RKLTKAEQQR FKEEAEMLKG LQHPNIVRFY DSWESILKGK KCIVLVTELM TSGTLKTYLK | 240 |
| RFKVMKPKVL RSWCRQILKG LQFLHTRTPP IIHRDLKCDN IFITGPTGSV KIGDLGLATL | 300 |
| MRTSFAKSVI GTPEFMAPEM YEEHYDESVD VYAFGMCMLE MATSEYPYSE CQNAAQIYRK | 360 |
| VTSGIKPASF NKVTDPEVKE IIEGCIRQNK SERLSIRDLL NHAFFAEDTG LRVELAEEDD | 420 |
| CSNSSLALRL WVEDPKKLKG KHKDNEAIEF SFNLETDTPE EVAYEMVKSG FFHESDSKAV | 480 |
| AKSIRDRVTP IKKTREKKPA GCLEERRDSQ CKSMGNVFPQ PQNTTLPLAP AQQTGAECEE | 540 |
| TEVDQHVRQQ LLQRKPQQHC SSVTGDNLSE AGAASVIHSD TSSQPSVAYS SNQTMGSQMV | 600 |
| SNIPQAEVNV PGQIYSSQQL VGHYQQVSGL QKHSKLTQPQ ILPLVQGQST VLPVHVLGPT | 660 |
| VVSQPQVSPL TVQKVPQIKP VSQPVGAEQQ AALLKPDLVR SLNQDVATTK ENVSSPDNPS | 720 |
| GNGKQDRIKQ RRASCPRPEK GTKFQLTVLQ VSTSGDNMVE CQLETHNNKM VTFKFDVDGD | 780 |
| APEDIADYMV EDNFVLESEK EKFVEELRAI VGQAQEILHV HFATERATGV DSITVDSNSS | 840 |
| QTGSSEQVQI NSTSTQTSNE SAPQSSPVGR WRFCINQTIR NRETQSPPSL QHSMSAVPGR | 900 |
| HPLPSPKNTS NKEISRDTLL TIENNPCHRA LFTSKSEHKD VVDGKISECA SVETKQPAIL | 960 |
| YQVEDNRQIM APVTNSSSYS TTSVRAVPAE CEGLTKQASI FIPVYPCHQT ASQADALMSH | 1020 |
| PGESTQTSGN SLTTLAFDQK PQTLSVQQPA MDAEFISQEG ETTVNTEASS PKTVIPTQTP | 1080 |
| GLEPTTLQPT TVLESDGERP PKLEFADNRI KTLDEKLRNL LYQEHSISSI YPESQKDTQS | 1140 |
| IDSPFSSSAE DTLSCPVTEV IAISHCGIKD SPVQSPNFQQ TGSKLLSNVA ASQPANISVF | 1200 |
| KRDLNVITSV PSELCLHEMS SDASLPGDPE AYPAAVSSGG AIHLQTGGGY FGLSFTCPSL | 1260 |
| KNPISKKSWT RKLKSWAYRL RQSTSFFKRS KVRQVETEEM RSAIAPDPIP LTRESTADTR | 1320 |
| ALNRCKAMSG SFQRGRFQVI TIPQQQSAKM TSFGIEHISV FSETNHSSEE AFIKTAKSQL | 1380 |
| VEIEPATQNP KTSFSYEKLQ ALQETCKENK GVPKQGDNFL SFSAACETDV SSVTPEKEFE | 1440 |
| ETSATGSSMQ SGSELLLKER EILTAGKQPS SDSEFSASLA GSGKSVAKTG PESNQCLPHH | 1500 |
| EEQAYAQTQS SLFYSPSSPM SSDDESEIED EDLKVELQRL REKHIQEVVN LQTQQNKELQ | 1560 |
| ELYERLRSIK DSKTQSTEIP LPPASPRRPR SFKSKLRSRP QSLTHVDNGI VATGKSCLIN | 1620 |
| ELENPLCVES NAASCQQSPA SKKGMFTDDL HKLVDDWTKE AVGNSLIKPS LNQLKQSQHK | 1680 |
| LETENWNKVS ENTPSTMGYT STWISSLSQI RGAVPTSLPQ GLSLPSFPGP LSSYGMPHVC | 1740 |
| QYNAVAGAGY PVQWVGISGT TQQSVVIPAQ SGGPFQPGMN MQAFPTSSVQ NPATIPPGPK | 1800 |
| Human WNK4 has the amino acid sequence (SEQ ID NO: 5): | |
| MLASPATETT VLMSQTEADL ALRPPPPIGT AGQPRLGPPP RRARRFSGKA EPRPRSSRLS | 60 |
| RRSSVDLGLL SSWSLPASPA PDPPDPPDSA GPGPARSPPP SSKEPPEGTW TEGAPVKAAE | 120 |
| DSARPELPDS AVGPGSREPL RVPEAVALER RREQEEKEDM ETQAVATSPD GRYLKFDIEI | 180 |
| GRGSFKTVYR GLDTDTTVEV AWCELQTRKL SRAERQRFSE EVEMLKGLQH PNIVRFYDSW | 240 |
| KSVLRGQVCI VLVTELMTSG TLKTYLRRFR EMKPRVLQRW SRQILRGLHF LHSRVPPILH | 300 |
| RDLKCDNVFI TGPTGSVKIG DLGLATLKRA SFAKSVIGTP EFMAPEMYEE KYDEAVDVYA | 360 |
| FGMCMLEMAT SEYPYSECQN AAQIYRKVTS GRKPNSFHKV KIPEVKEIIE GCIRTDKNER | 420 |
| FTIQDLLAHA FFREERGVHV ELAEEDDGEK PGLKLWLRME DARRGGRPRD NQAIEFLFQL | 480 |
| GRDAAEEVAQ EMVALGLVCE ADYQPVARAV RERVAAIQRK REKLRKAREL EALPPEPGPP | 540 |
| PATVPMAPGP PSVFPPEPEE PEADQHQPFL FRHASYSSTT SDCETDGYLS SSGFLDASDP | 600 |
| ALQPPGGVPS SLAESHLCLP SAFALSIPRS GPGSDFSPGD SYASDAASGL SDVGEGMGQM | 660 |
| RRPPGRNLRR RPRSRLRVTS VSDQNDRVVE CQLQTHNSKM VTFRFDLDGD SPEEIAAAMV | 720 |
| YNEFILPSER DGFLRRIREI IQRVETLLKR DTGPMEAAED TLSPQEEPAP LPALPVPLPD | 780 |
| PSNEELQSST SLEHRSWTAF STSSSSPGTP LSPGNPFSPG TPISPGPIFP ITSPPCHPSP | 840 |
| SPFSPISSQV SSNPSPHPTS SPLPFSSSTP EFPVPLSQCP WSSLPTTSPP TFSPTCSQVT | 900 |
| LSSPFFPPCP STSSFPSTTA APLLSLASAF SLAVMTVAQS LLSPSPGLLS QSPPAPPSPL | 960 |
| PSLPLPPPVA PGGQESPSPH TAEVESEASP PPARPLPGEA RLAPISEEGK PQLVGRFQVT | 1020 |
| SSKEPAEPLP LQPTSPTLSG SPKPSTPQLT SESSDTEDSA GGGPETREAL AESDRAAEGL | 1080 |
| GAGVEEEGDD GKEPQVGGSP QPLSHPSPVW MNYSYSSLCL SSEESESSGE DEEFWAELQS | 1140 |
| LRQKHLSEVE TLQTLQKKEI EDLYSRLGKQ PPPGIVAPAA MLSSRQRRLS KGSFPTSRRN | 1200 |
| SLQRSEPPGP GIMRRNSLSG SSTGSQEQRA SKGVTFAGDV GRM | 1243 |

In addition, WNKs stimulate the kinases SPAK and OSR1, which directly phosphorylate and stimulate Cl(−)-importing, Na(+)-driven CCCs or inhibit the Cl(−)-extruding, K(+)-driven cation-chloride cotransporters (CCCs). These coordinated and reciprocal actions on the CCCs are triggered by an interaction between RFXV/I motifs within the WNKs and CCCs and a conserved carboxyl-terminal docking domain in SPAK and OSR1. Human STE20/SPS1-related proline-alanine-rich protein kinase (SPAK) has the sequence (SEQ ID NO: 6):

| | |
|---|---|
| MAEPSGSPVH VQLPQQAAPV TAAAAAAPAA ATAAPAPAAP AAPAPAPAPA AQAVGWPICR | 60 |
| DAYELQEVIG SGATAVVQAA LCKPRQERVA IKRINLEKCQ TSMDELLKEI QAMSQCSHPN | 120 |
| VVTYYTSFVV KDELWLVMKL LSGGSMLDII KYIVNRGEHK NGVLEEAIIA TILKEVLEGL | 180 |
| DYLHRNGQIH RDLKAGNILL GEDGSVQIAD FGVSAFLATG GDVTRNKVRK TFVGTPCWMA | 240 |
| PEVMEQVRGY DFKADMWSFG ITAIELATGA APYHKYPPMK VLMLTLQNDP PTLETGVEDK | 300 |
| EMMKKYGKSF RKLLSLCLQK DPSKRPTAAE LLKCKFFQKA KNREYLIEKL LTRTPDIAQR | 360 |
| AKKVRRVPGS SGHLHKTEDG DWEWSDDEMD EKSEEGKAAF SQEKSRRVKE ENPEIAVSAS | 420 |
| TIPEQIQSLS VHDSQGPPNA NEDYREASSC AVNLVLRLRN SRKELNDIRF EFTPGRDTAD | 480 |
| GVSQELFSAG LVDGHDVVIV AANLQKIVDD PKALKTLTFK LASGCDGSEI PDEVKLIGFA | 540 |
| QLSVS | 545 |

Human Serine/threonine-protein kinase OSR1 (OSR1, gene name: OXSR1) has the sequence (SEQ ID NO: 7):

| | |
|---|---|
| MSEDSSALPW SINRDDYELQ EVIGSGATAV VQAAYCAPKK EKVAIKRINL EKCQTSMDEL | 60 |
| LKEIQAMSQC HHPNIVSYYT SFVVKDELWL VMKLLSGGSV LDIIKHIVAK GEHKSGVLDE | 120 |
| STIATILREV LEGLEYLHKN GQIHRDVKAG NILLGEDGSV QIADFGVSAF LATGGDITRN | 180 |
| KVRKTFVGTP CWMAPEVMEQ VRGYDFKADI WSFGITAIEL ATGAAPYHKY PPMKVLMLTL | 240 |
| QNDPPSLETG VQDKEMLKKY GKSFRKMISL CLQKDPEKRP TAAELLRHKF FQKAKNKEFL | 300 |
| QEKTLQRAPT ISERAKKVRR VPGSSGRLHK TEDGGWEWSD DEFDEESEEG KAAISQLRSP | 360 |
| RVKESISNSE LFPTTDPVGT LLQVPEQISA HLPQPAGQIA TQPTQVSLPP TAEPAKTAQA | 420 |
| LSSGSGSQET KIPISLVLRL RNSKKELNDI RFEFTPGRDT AEGVSQELIS AGLVDGRDLV | 480 |
| IVAANLQKIV EEPQSNRSVT FKLASGVEGS DIPDDGKLIG FAQLSIS | 527 |

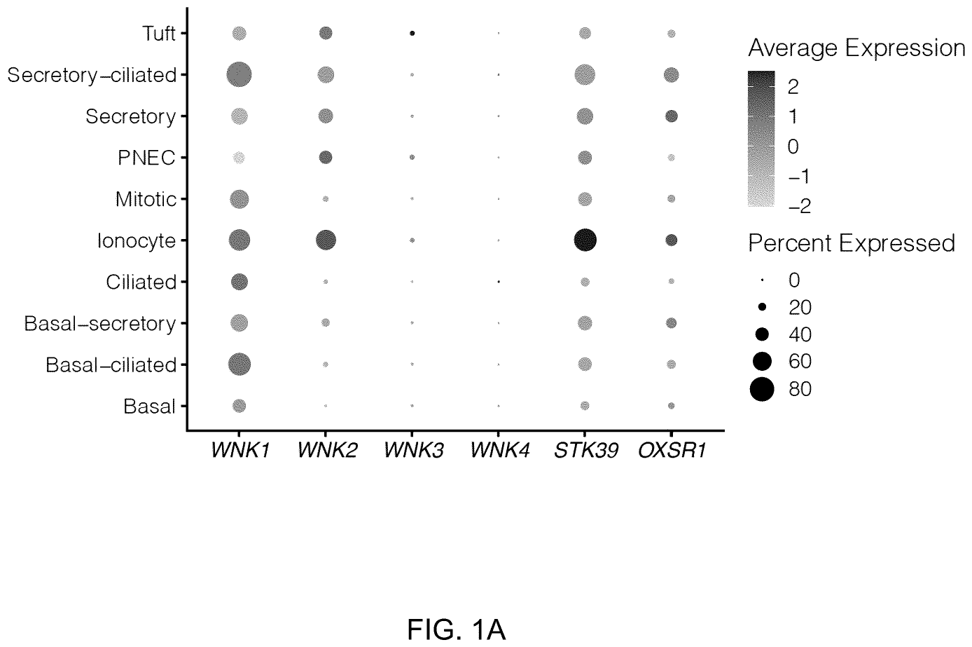

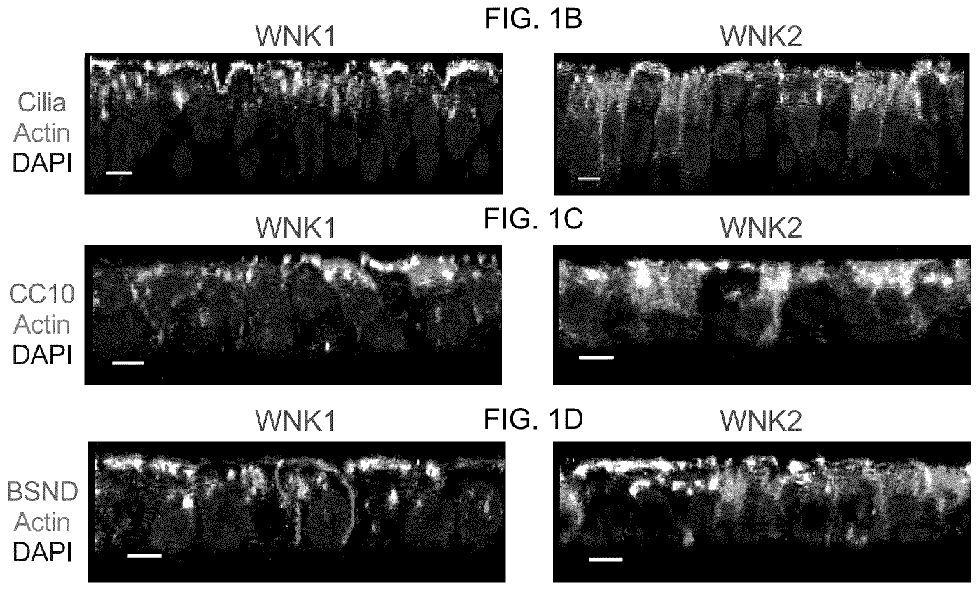

As used herein, “WNK kinase pathway inhibitor” refers to a compound or composition that inhibits the biological activity of a member of the WNK kinase pathway. Exemplary members of the WNK kinase pathway include, but are not limited to, with-no-lysine kinase 1, 2, 3, and 4 (WNK1, WNK2, WNK3, and WNK4), Serine/threonine-protein kinase OSR1 (OSR1), and Ste20/SPS1-related proline-alanine-rich protein kinase (SPAK). WNK1, 2, 3, and 4, OSR1, and SPAK each have the enzyme activity EC: 2.7.11.1, or kinase activity. Therefore, in some embodiments, a WNK kinase pathway inhibitor inhibits the EC: 2.7.11.1 activity, or kinase activity, of one or more of WNK1, 2, 3, 4, OSR1, and SPAK. The inventors discovered that both WNK1 and WNK2 were expressed in cells of the airway epithelium (FIG. 1A). However, WNK2 was expressed in secretory cells and ionocytes, which are predominantly responsible for regulating ASL pH (FIG. 1A). Thus, in some embodiments, the WNK pathway inhibitor is an inhibitor of WNK1 or WNK2. In some embodiments, the WNK pathway inhibitor is a selective inhibitor of WNK2.

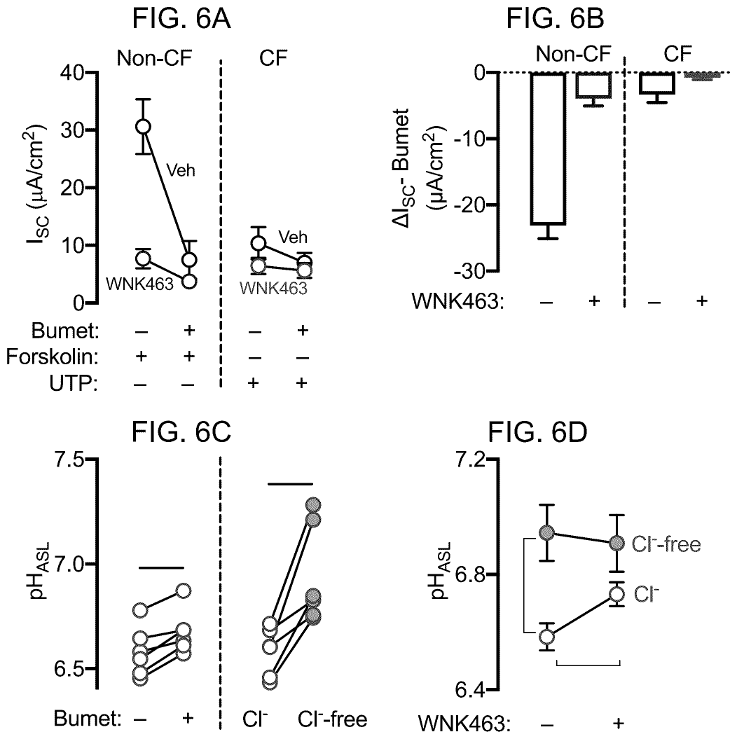

The inventors discovered that WNK pathway inhibitors reduce Na—K-2Cl cotransporter-1 (NKCC1) activity (FIG. 6D). Therefore, in some embodiments, the methods reduce the activity of NKCC1.

Exemplary WNK pathway inhibitors are known in the art; however, it is to be understood that the instantly disclosed methods are not limited to the following exemplary WNK pathway inhibitors.

Exemplary WNK inhibitors include, but are not limited to:

WNK463, which has the formula:

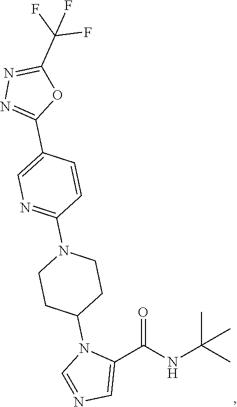 [SMILES: CC(C)(C)NC(=O)c1cncn1C1CCN(c2ccc(-c3nnc(C(F)(F)F)o3)cn2)CC1]

WNK-IN-11, which has the formula:

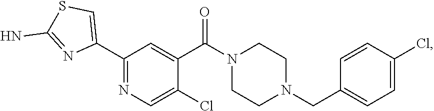 [SMILES: Nc1nc(-c2cc(C(=O)N3CCN(Cc4ccc(Cl)cc4)CC3)c(Cl)cn2)cs1]

and STOCK2S-26016, which has the formula:

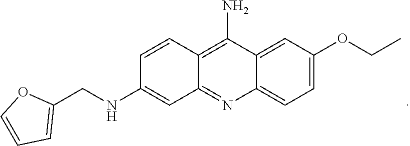 [SMILES: CCOc1ccc2nc3cc(NCc4ccco4)ccc3c(N)c2c1]

Exemplary inhibitors of SPAK and OSR1 include, but are not limited to:

Rafoxanide, which has the formula:

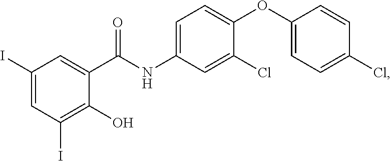 [SMILES: O=C(Nc1ccc(Oc2ccc(Cl)cc2)c(Cl)c1)c1cc(I)cc(I)c1O]

and

Closantel, which has the formula:

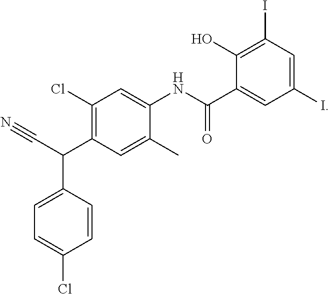 [SMILES: Cc1cc(C(C#N)c2ccc(Cl)cc2)c(Cl)cc1NC(=O)c1cc(I)cc(I)c1O]

In some embodiments, a composition of the present disclosure is administered to a subject according to a schedule as determined by one of skill in the art (as discussed previously), taking into consideration clinical and/or diagnostic variables particular to that subject. In some embodiments, the composition is administered once per day, every other day, twice per day. In some embodiments, the composition is administered for 1, 2, 3, 4, 5 6, 7, 8, 9, 10, 11, 12, 13, days, two weeks, 3 weeks, 4 week, 5 weeks, 6 weeks, 3 months, 6 months, 1 year or more.

In another aspect of the current disclosure, methods of treating one or more signs or symptoms of cystic fibrosis in a subject in need thereof are provided. In some embodiments, the methods comprise: administering to the subject an effective amount of a WNK kinase pathway inhibitor to the subject to treat the one or more signs or symptoms of cystic fibrosis.

Exemplary signs and symptoms of cystic fibrosis comprise one or more of A persistent cough that produces thick mucus (sputum), wheezing, exercise intolerance, repeated lung infections, inflamed nasal passages or a stuffy nose, and recurrent sinusitis.

In another aspect of the current disclosure, methods of increasing the pH of airway surface liquid (ASL) in a subject in need thereof are provided. In some embodiments, the methods comprise: administering to the subject an effective amount of a WNK kinase pathway inhibitor to increase the pH of the ASL in the subject.

Cystic fibrosis (CF) is characterized by abnormally low pH of the ASL, or a pH of less than about 6.9 to about 7.1. Therefore, in some embodiments, treatment of cystic fibrosis comprises increasing the ASL pH about 0.1, about 0.2, about 0.3, or more to result in a relatively normal ASL pH of about 6.9 to about 7.1.

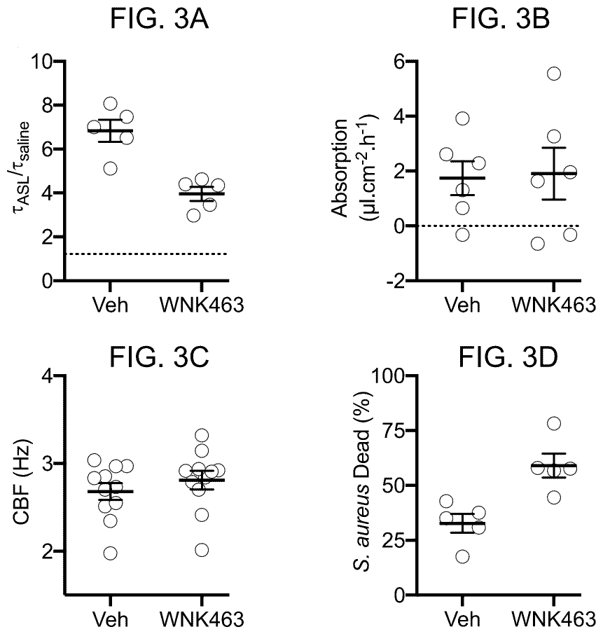

Related to the abnormal pH of airway fluids, subjects suffering from CF have increased incidence of infection in the airway. Accordingly, in some embodiments, treatment of CF comprises reducing the frequency of airway infections in a subject. The inventors demonstrated that treatment of primary airway cultures from subjects with CF with a WNK pathway inhibitor increased the ASL-mediated killing of S. aureus (FIG. 3D). Thus, in some embodiments, the disclosed methods increase killing, or reduce the survival, of pathogenic or commensal bacteria in the airway.

Therefore, in another aspect of the current disclosure, methods of enhancing respiratory defense in a subject in need thereof are provided. In some embodiments, the methods comprise: administering to the subject an effective amount of a WNK kinase pathway inhibitor to enhance respiratory defense in the subject.

As used herein, “enhancing respiratory defense in a subject” refers to increasing the ability of the subject to kill, or reduce the rate of growth of microorganisms, e.g., viruses, bacteria, fungi, etc., in the respiratory tract of the subject. As discussed above, the inventors discovered that WNK kinase pathway inhibitor treatment increased the ability of primary lung cells to kill S. aureus in vitro (FIG. 3D). Methods of determining killing of microbes and the relative ability of microbial cells to grow on ASL substrates are known in the art.

Methods of Testing a Compound for Use in Treating Cystic Fibrosis

In another aspect of the current disclosure, methods of testing a compound of interest for use as a treatment for cystic fibrosis are provided. In some embodiments, the methods comprise: (a) culturing cells in the presence and absence of the compound of interest; (b) culturing control cells in the presence and absence of a WNK kinase pathway inhibitor; (c) detecting one or more parameters related to lung airway function in the cultured cells of step (a) and the cultured cells of step (b); (d) generating a test index by calculating the change in the one or more parameters between the cultured cells of step (a) in the presence and absence of the compound of interest; and (e) generating a control index by calculating the change in the one or more parameters between the cultured cells of step (b) in the presence and absence of the WNK kinase pathway inhibitor; wherein if the value of the test index is equal to, or improved, as compared to the value of the control index, then the compound of interest is of use for the treatment of cystic fibrosis.

Exemplary cells for use in the methods of testing a compound include, but are not limited to, differentiated primary human airway epithelial cells, NuLi-1 cells, or CuFi-4 cells. Primary human airway epithelial cells may be differentiated by methods known in the art. For example, primary cultures of differentiated airway epithelia may be obtained without passage from multiple human donors as previously reported in Karp et al. (Karp P H, Moninger T O, Weber S P, Nesselhauf T S, Launspach J L, Zabner J, et al. An in vitro model of differentiated human airway epithelia. Methods for establishing primary cultures. Methods Mol Biol. 2002; 188:115-37). Briefly, proximal bronchi are dissected, cut into small pieces, and enzymatically digested. Epithelial cells are isolated and seeded onto collagen-coated inserts (Costar, 3470; Falcon, 353180). Epithelia are differentiated at the air liquid interface for 3 weeks or more prior to use in an assay. As an alternative, epithelial cells from previous CF donors cryopreserved at PO may be thawed and differentiated. Whenever feasible, assays should utilize a paired design so that epithelia from the same donor are assayed under control (absence of compound of interest) and treatment (presence of compound of interest) conditions. As yet another alternative, differentiated airway epithelia may be generated from cryopreserved transformed human airway epithelial cell lines NuLi-1 (WT/WT) and CuFi-4 (G551D/ΔF508), as previously reported (Zabner J, Karp P, Seiler M, Phillips S L, Mitchell C J, Saavedra M, et al. Development of cystic fibrosis and noncystic fibrosis airway cell lines. Am J Physiol Lung Cell Mol Physiol. 2003; 284(5):L844-54).

The disclosed methods of testing a compound comprise detecting one or more parameters related to lung airway function in cells cultured in the presence and absence of the compound of interest. Exemplary parameters related to lung airway function in cells include, but are not limited to, pH, HCO₃⁻ secretion, Cl⁻ secretion, mucus viscosity, bacterial colonization, and electrical conductance across the membrane of the cells. As will be clear to one of skill in the art, the foregoing factors are intended to reflect measurable or quantifiable aspects of the function of airway epithelial cells. Therefore, additional measurable parameters are contemplated by the instant disclosure, so long as they reflect a function of airway epithelial cells. Suitably, the measured parameter is defective in airway epithelial cells with mutations in CFTR, e.g., primary cells from a subject with cystic fibrosis, e.g., a subject with a mutation in CFTR selected from the mutations listed in Table 1. Thus, if the parameter is altered in the presence of the compound of interest or, for example, returned to “wild type levels”, then the compound of interest is of use in the treatment of cystic fibrosis. As used herein, “wild type levels” of a parameter refers to the level of a parameter in control or wild type cells, e.g., cells without a mutation in CFTR or in any other known protein that affects the function of the cells.

Certain parameters are increased in cells with mutations in CFTR, e.g., mucus viscosity, bacterial colonization, whereas certain parameters are decreased, e.g., pH. Thus, if, in the case of bacterial colonization as the selected parameter, the compound of interest reduces bacterial colonization to a similar degree as the WNK pathway inhibitor, then the compound of interest is of use in the treatment of cystic fibrosis. Likewise, if, in the case of pH, the compound of interest increases pH to a similar degree as the WNK pathway inhibitor, then the compound of interest is of use in the treatment of cystic fibrosis. Use of the phrase “equal to or improved as compared to the control index” is intended to reflect that some parameters are increased in CF cells, while others are decreased, e.g., pH. Thus, “improved” refers to a change in the selected parameter greater than, but in the same direction, as the WNK pathway inhibitor, when compared to cells cultured in the absence of WNK pathway inhibitors or the compound of interest.

In some embodiments, the disclosed method of testing a compound comprises administering an effective amount of the compound of interest to a subject in need thereof to treat the cystic fibrosis.

Miscellaneous

The phrases “% sequence identity,” “percent identity,” or “% identity” refer to the percentage of amino acid residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofamesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain.

Nucleic acids, proteins, and/or other compositions described herein may be purified. As used herein, “purified” means separate from the majority of other compounds or entities, and encompasses partially purified or substantially purified. Purity may be denoted by a weight by weight measure and may be determined using a variety of analytical techniques such as but not limited to mass spectrometry, HPLC, etc.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides, (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “hybridization”, as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning-A Laboratory Manual, Cold Spring Harbor Laboratory, Cold Spring Harbor, New York; Wetmur, 1991, Critical Review in Biochem. and Mol. Biol. 26(3/4):227-259; and Owczarzy et al., 2008, Biochemistry, 47: 5336-5353, which are incorporated herein by reference).

As used herein the term “effective amount” refers to the amount or dose of the compound, upon single or multiple dose administration to the subject, which provides the desired effect in the subject under diagnosis or treatment.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

Intrapulmonary administration, e.g., administration by inhalation, is an illustrative route of administering the compounds employed in the compositions and methods disclosed herein. Without being limited by any theory or mechanism, the inventors believe that intrapulmonary administration of WNK pathway inhibitors in the disclosed methods may increase efficacy and reduce side effects by administering the WNK pathway inhibitors directly to tissue in need of WNK pathway inhibition, i.e., the airway epithelium. Other illustrative routes of administration include oral, transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

### EXAMPLES

The following Examples are illustrative and should not be interpreted to limit the scope of the claimed subject matter.

### Example 1—WNK Inhibition Increases Surface Liquid pH and Host Defense in Cystic Fibrosis Airway Epithelia

### INTRODUCTION

Cystic fibrosis (CF) is an inherited, multisystem channelopathy caused by mutations in the cystic fibrosis transmembrane conductance regulator (CFTR) gene (1-3). Loss of CFTR protein function reduces anion secretion, disrupts epithelial function, and impairs airway host defense. These abnormalities result in chronic airway obstruction, inflammation, infection, tissue destruction and bronchiectasis, and limit the life span of affected individuals.

CFTR is an apical HCO₃⁻ and Cl⁻ channel (4-6). In airway epithelia, these transport activities control the acid-base balance and composition of the thin film of liquid, the airway surface liquid (ASL), that covers the apical membrane. The ASL interfaces with the environment and mediates at least two vital respiratory host defenses (7-9). Mucociliary clearance uses gel-forming mucins to trap inhaled particles and ciliary beating to propel them out of the airways. Secreted antimicrobial peptides disrupt bacterial cell membranes and kill inhaled pathogens. An abnormally acidic pH of the ASL (pH_{ASL}) resulting from reduced CFTR-mediated HCO₃⁻ secretion impairs these respiratory defenses (10-18). Importantly, ASL alkalinization rescues these defects and may benefit CF individuals independent of CFTR genotype (19-21).

Transepithelial HCO₃⁻ secretion is a complex process. Several studies have identified key apical and basolateral transporters involved in this process (11, 22-25); others have resolved tissue-specific and species-specific differences (26-28). However, the cellular and molecular mechanisms that regulate airway HCO₃⁻ secretion in humans remain incompletely defined. CF airways express apical HCO₃⁻ channels and transporters other than CFTR (8, 29). Thus, identifying mechanisms that regulate non-CFTR HCO₃ secretion may suggest novel ways to raise CF pH_{ASL}.

We considered that knowledge of HCO₃⁻ transport in non-airway epithelia might yield insights relevant to CF airways. In several epithelia, the with-no-lysine [K] (WNK) kinases act as key regulators of anion transport (30, 31). WNK kinases are serine/threonine protein kinases that modify surface expression or activity of membrane transporters. In the pancreas, which shares similarities with airway HCO₃⁻ transport, WNK kinases control ductal HCO₃⁻ secretion (26, 32). In one study of mouse pancreatic duct, silencing of WNK kinases increased, and WNK expression decreased HCO₃⁻ secretion (33). In other reports, these kinases were shown to modulate CFTR HCO₃⁻ channel activity (34, 35) and membrane expression of SLC26 family transporters (36). However, whether WNK kinases coordinate HCO₃⁻ secretion across human airway epithelia remains poorly understood.

In this study, we tested the hypothesis that WNK kinases regulate CF pH_{ASL}. We studied primary cultures of differentiated human airway epithelia, and applied pharmacologic and genetic interventions to elicit responses. Our results show that airway epithelia express two WNK isoforms, WNK1 and WNK2, in secretory cells and ionocytes. Importantly, reducing WNK kinase activity increases pH_{ASL} and enhances key respiratory host defenses that are otherwise impaired in CF.

Methods

Cell culture. Primary cultures of differentiated airway epithelia were obtained without passage from multiple human donors as previously reported (37). Briefly, proximal bronchi were dissected, cut into small pieces, and enzymatically digested. Epithelial cells were isolated and seeded onto collagen-coated inserts (Costar, 3470; Falcon, 353180). Epithelia were differentiated at the air liquid interface for 3 weeks or more prior to assay. During the course of this study, new CF lung donors became scarce, partly due to more individuals taking highly-active CFTR modulators. To manage this situation, epithelial cells from previous CF donors cryopreserved at PO were thawed and differentiated. Whenever feasible, studies followed a paired design so that epithelia from the same donor were assayed under control and treatment conditions.

In some experiments, differentiated airway epithelia were generated from cryostocks of transformed human airway epithelial cell lines NuLi-1 (WT/WT) and CuFi-4 (G551D/ΔF508), as previously reported (38).

To assess cytokine-induced responses, epithelia were treated with a combination of 10 ng/ml TNFα (R&D Systems) and 20 ng/ml IL-17 (R&D Systems). Both cytokines were added to the basolateral media for 48 hours prior to assessments. All studies were approved by the University of Iowa Institutional Review Board.

Pharmacologic reagents. WNK463 was purchased from Selleckchem. Other reagents were purchased from MilliporeSigma.

Single Cell RNA-seg and analysis. Libraries were generated for single cell RNA-seq according to the Chromium Single Cell Gene Expression v3 kit 10X Genomics protocol (10X Genomics, Pleasanton, CA). Cells were combined with Gel Beads, Master Mix, and Partitioning Oil, and loaded onto a Chromium Next GEM Chip with a target of 5,000 cells per sample. Single cells were partitioned in oil to generate gel beads in emulsion (GEMs). GEMs were dissolved and barcoded with Illumina TruSeq sequencing primer, barcode, and unique molecular identifier (UMI), and full-length cDNA was generated via reverse transcription. After an additional round of cDNA amplification, enzymatic fragmentation was used to select the appropriate amplicon size and then labeled via End Repair, A-tailing, Adaptor Ligation, and PCR to construct libraries for sequencing. Sequencing was performed on the HiSeq or NovaSeq 6000 μlatform.

For bioinformatics analysis, CellRanger software version 3.0.2 was used to parse cell barcodes and UMIs, and align raw sequencing reads to human genome reference GRCh38.p13. Gene-by-cell count matrices were analyzed using the R package Seurat version 3.1.1 (39). Counts for each cell were normalized by total UMIs and log transformed to quantify gene expression for each cell. To reduce the data dimensionality for clustering and visualization, centered and scaled gene expression for the 2,000 mostly highly variable genes were further reduced to the first 20 principal component scores for input to a shared nearest neighbor clustering algorithm. Cell type identities were associated with each cluster by identifying upregulated genes in each cluster using a Wilcoxon rank sum test and comparing upregulated genes to a list of known airway epithelial markers. For plots of WNK gene expression, cells were stratified by cell type and donor, and gene counts were summed across cells, resulting in a gene-by-donor count matrix for each cell type. The data are available in the NCBI's GEO database (GEO GSE159056).

Immunocytochemistry. Airway epithelia were washed 3 times with PBS, fixed with 4% paraformaldehyde for 15 minutes, and permeabilized with 0.3% Triton-X for 20 minutes. To minimize nonspecific staining, epithelia were treated with SuperBlock (Thermo Fisher Scientific) containing 0.5% normal goat serum for 1 hour at room temperature. Primary antibodies were diluted in SuperBlock and added apically for 3 hours at 37° C. Epithelia were washed and incubated for 45 minutes with appropriate secondary antibodies diluted in PBS. The following primary antibodies were used: rabbit anti-WNK1 (1:200; Cell Signaling Technology cat. no. 4979); rabbit anti-WNK2 (1:200; Novus Biologicals cat. no. NBP2-33875); mouse anti-acetyl-α-tubulin (1:200, Invitrogen cat. no. 32-270-0); mouse anti-CC10 (1:200; Santa Cruz Biotechnology cat. no. sc-365992); and mouse anti-BSND (1:200; Abnova cat. no. H00007809⁻BO1P). To detect primary antibodies, the following secondary antibodies were used: goat anti-rabbit conjugated to Alexa Flour 488 (1:1000; Thermo Fisher Scientific cat. no. A11070), and goat anti-mouse conjugated to Alexa Flour 568 (1:1000; Thermo Fisher Scientific cat. no. A11019). Actin cytoskeleton was stained with Alexa Fluor 633 phalloidin (1:300; Thermo Fisher Scientific cat. no. A22284) added at the same time as secondary antibodies. Epithelia were mounted on glass slides, and coverslips were secured using Vectashield with DAPI (Vector Laboratories). Imaging was performed on the Olympus Fluoview FV 3000 confocal microscope. Z-stack images were processed using the Olympus Fluoview program.

pH_{ASL} measurement. pH_{ASL} was measured using a fluorescent ratiometric pH indicator, SNARF-1, conjugated to 70 kD dextran (Thermo Fisher Scientific). SNARF-1-dextran was delivered as a powder to the apical side and allowed to distribute into ASL for 1 hour. Imaging was performed on a laser-scanning confocal microscope (Zeiss LSM 880). SNARF-1 was excited at 514 nm, and emissions at 580 nm and 640 nm were recorded. The ratio of fluorescence intensities (580/640) was obtained to calculate pH values using calibration curves constructed from colorless standard pH solutions. The microscope chamber housing epithelia maintained a humidified environment at 37° C. 5% CO₂ was added to the chamber environment whenever the basolateral side was immersed in a HCO₃⁻ containing buffer solution, but removed when a HCO₃⁻-free (HEPES) buffer solution was used.

ASL viscosity measurement. ASL viscosity was measured using the fluorescence recovery after photobleaching (FRAP) method as previously reported (12). Briefly, powdered FITC-dextran (70 kD, Sigma) was delivered to the apical side of airway epithelia, and allowed to equilibrate for 2 hours. Epithelia were moved to a humidified chamber on the Zeiss LSM 880 confocal microscope. The chamber maintained a temperature of 37° C. and an atmosphere of 5% CO₂. After baseline imaging, a small region of interest was photobleached. Time series fluorescence recovery images were collected until maximal recovery was reached. For each epithelium, 4-5 curves from different locations were obtained and averaged. The time constant (T) was calculated from the fluorescence recovery curves using regression, and expressed as ASL value normalized to saline (τ_{ASL}/τₛₐₗᵢₙₑ).

Liquid absorption assay. Liquid absorption was measured using a previously reported method (40). Briefly, airway epithelia were transferred to 500 μl of fresh basolateral solution containing either vehicle or 10 μM WNK463. The apical side was suctioned dry and 30 μl of Krebs buffer solution was introduced. Of note, the apical solution had the same ionic composition, osmolality, and pH as the basolateral solution. After 4 hours, the apical liquid was collected with micropipettes (Drummond Microcaps #1-000-1000) and the height of the liquid column was recorded. Volume of liquid was calculated as the product of height and micropipette cross-sectional area. The difference between the original and 4-hour liquid volumes was used to calculate the rate of absorption (μl·cm⁻²·h⁻¹).

Ciliary beat frequency measurement. Phase contrast videos of ciliary motion were obtained using the Zeiss Axio Observer microscope at 50 frames per second at room temperature. A total of 256 frames (5.1 second videos) were obtained for each field. A minimum of five random fields per culture were selected. Whole field gaussian mean ciliary beat frequencies (CBF) were analyzed using the Sisson-Ammons video analysis software (SAVA; Ammons Engineering, Mt. Morris, MI) (41).

S. aureus killing assay. ASL antimicrobial activity was assessed using bacteria-coated grids. Preparation, imaging, and quantification were performed as previously described (13). Briefly, S. aureus isolate SA-CL37 was grown to log-phase, labeled with biotin, and conjugated to streptavidin-coated gold electron microscopy grids. The grids were placed on the apical side of airway epithelia in direct contact with ASL. After incubating for 15 minutes in humidified, 5% CO₂ chambers at 37° C., the grids were retrieved, rinsed with PBS, and immersed in SYTO9 and propidium iodide to determine bacterial viability (Live/Dead Bacterial Viability Assay, Invitrogen). The grids were washed, fixed with 4% paraformaldehyde, and imaged on the Olympus Fluoview FV 3000 confocal microscope. Images were analyzed for numbers of live and dead bacteria using the ImageJ software. For each grid, three separate regions were imaged. Two grids were assessed per epithelium and the results averaged to obtain % S. aureus killing.

siRNA knockdown. Gene knockdown in primary CF airway epithelia was achieved as reported previously (42). siRNAs were obtained from Integrated DNA Technologies (negative control: IDT DS NC 1; WNK1: IDT hs.Ri.WNK1.13.2; WNK2: IDT hs.Ri.WNK2.13.3) and transfected into dissociated primary airway epithelial cells using Lipofectamine RNAiMax (Invitrogen). Transfected cells were seeded onto collagen-coated inserts (Costar, 3470) and differentiated at the air-liquid interface. pH_{ASL} was measured at day 6 or 7 after seeding. The efficiency of gene knockdown was assessed with RT-PCR.

Electrophysiologic studies. Airway epithelia were mounted in modified Ussing chambers (Physiologic Instruments) and bathed in symmetric Krebs buffer solution. The standard Krebs buffer contained (in mM): 118.9 NaCl, 25 NaHCO₃, 2.4 K₂HPO₄, 0.6 KH₂PO₄, 1.2 MgCl₂, 1.2 CaCl₂), and 5 dextrose at 37° C., adjusted to pH 7.4 in the presence of 5% CO₂. The HCO₃⁻-free solution contained (in mM): 135 NaCl, 2.4 K₂HPO₄, 0.6 KH₂PO₄, 1.2 MgCl₂, 1.2 CaCl₂), 5 HEPES, and 5 dextrose at 37° C., adjusted to pH 7.4 in nominally C02⁻free environment. The Cl⁻-free solution contained (in mM): 118.9 Na gluconate, 25 NaHCO₃, 2.4 K₂HPO₄, 0.6 KH₂PO₄, 5 Ca gluconate, 1 Mg gluconate, 5 dextrose, at 37° C. and adjusted to pH 7.4 in the presence of 5% C02. Epithelia were voltage clamped, followed by recording of the short-circuit current (I_{SC}) and transepithelial conductance (Gₜ). Exposure to either vehicle (DMSO) or WNK463 was continued during recording. The following agents were added apically (in μM): 100 amiloride, 20 uridine triphosphate (UTP), 100 4,4′-diisothiocyano-2,2′-stilbenedisulfonic acid (DIDS), 10 forskolin, and 10 CFTRᵢₙₕ-172. In some experiments, 100 μM bumetanide was also added to the serosal chambers.

Bulk RNA-seg. RNA isolation, library preparation, sequencing, and bioinformatics analysis were previously reported (43). RNA-seq data are available in the NCBI's GEO database (GEO GSE176121).

Real-time PCR. Total RNA was isolated from airway epithelia using the RNeasy Lipid Tissue Mini Kit (QIAGEN). Genomic DNA was removed through DNase I (QIAGEN) treatment. RNA quality was verified using NanoDrop 2000 spectrophotometer (Thermo Fisher Scientific), and samples with a 260:280 ratio ≥1.8 were carried forward. RNA was reverse transcribed using the SuperScript VILO MasterMix (Invitrogen). Amplification was performed using gene-specific primers and Fast SYBR Green Master Mix (Applied Biosystems) on the QuantStudio6Pro Real-Time PCR System (Applied Biosystems). The primer pairs used were as follows: WNK1, 5′-GCCGTCAGATCCTTAAAGGTC-3′ (SEQ ID NO: 8) and 5′-CCAGTAGGGCCGGTGATAA-3′ (SEQ ID NO: 9); WNK2, 5′-CATACCTGAAGCGGTTCAAGG-3′ (SEQ ID NO: 10) and 5′-CTTTTGGCAAATGACGCTCTTT-3′ (SEQ ID NO: 11); and SFRS9, 5′-TGCGTAAACTGGATGACACC-3′ (SEQ ID NO: 12) and 5′-CCTGCTTTGGTATGGAGAGTC-3′ (SEQ ID NO: 13). All reactions were performed in triplicates. Gene expression was quantitated using −ΔΔCT method.

Statistics. Statistical significance testing was performed on GraphPad Prism 8 Software. Statistical tests included paired Student's t test for comparing 2 groups, and one-way ANOVA with Tukey's multiple comparison test for comparing more than 2 groups. A P value of less than 0.05 was considered significant.

### Results

Airway epithelia express WNK1 and WNK2. The four WNK isoforms are expressed in a tissue-specific manner (44, 45). However, their expression in human airways remains relatively unexplored. Recent scRNA-seq studies have revealed considerable cellular-level heterogeneity within airway epithelia with implications for ion transport (46-48). Notably, these studies have shown that secretory cells express nearly half the epithelial CFTR transcript, and the ionocytes, though rare, express the highest levels on a per cell basis (46-49). These cell types also express basolateral transporters involved in HCO₃⁻ and Cl⁻ secretion.

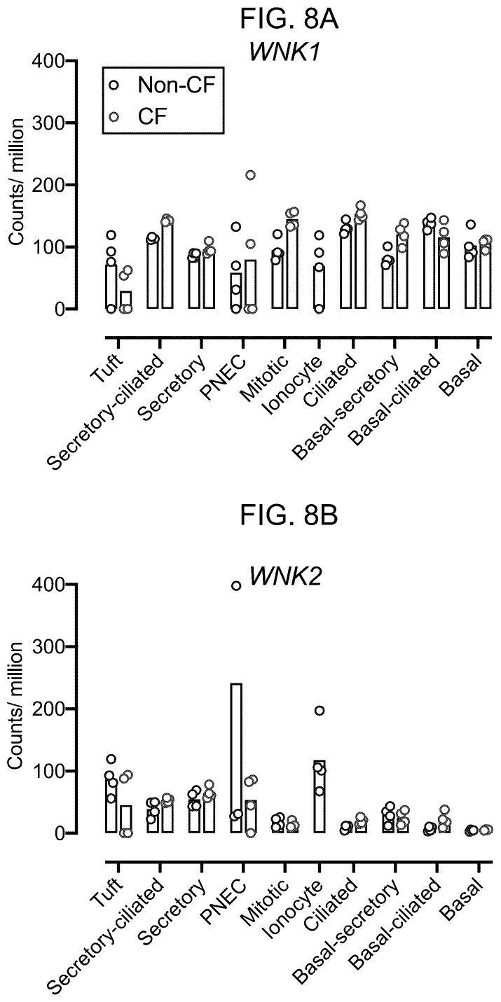

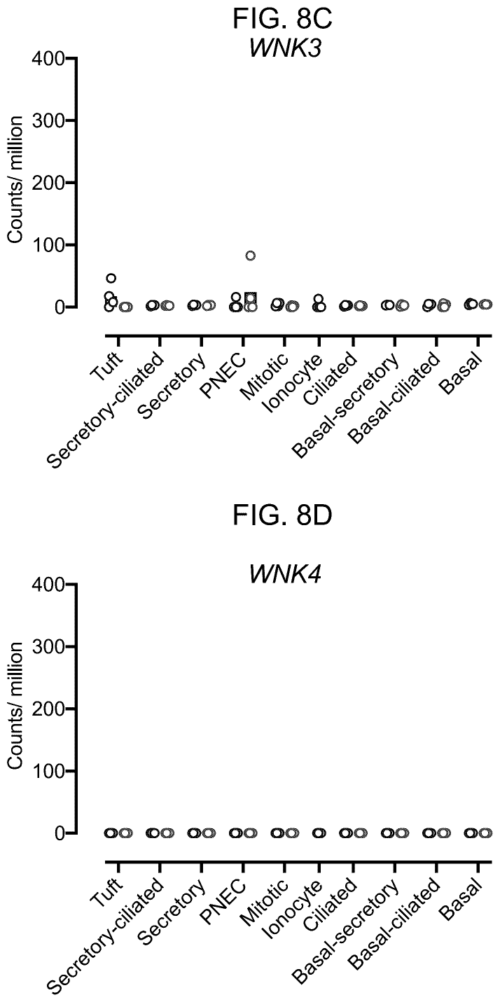

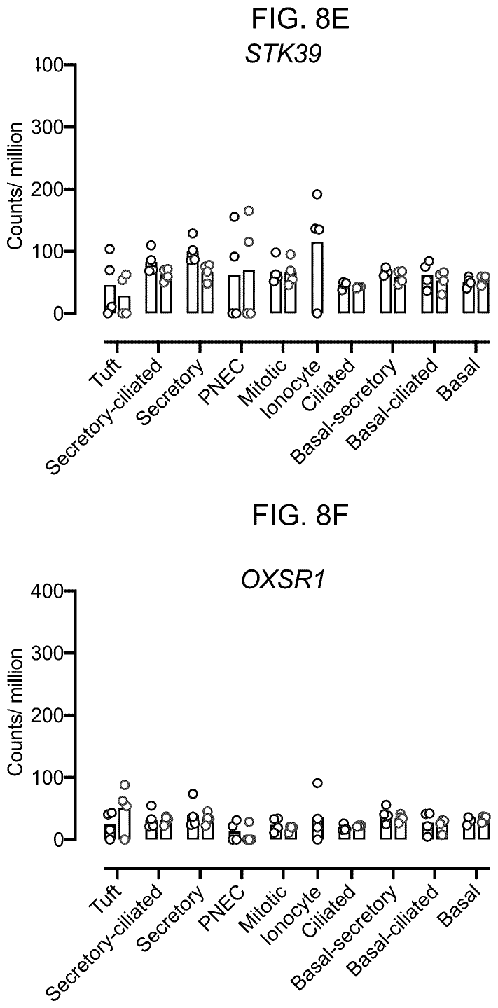

To identify which WNK kinases might regulate anion transport across airway epithelia, we performed scRNA-seq. We studied primary cultures of differentiated airway epithelia from four different CF and non-CF donors, and studied cell type-specific WNK gene expression (FIG. 1A). WNK1 was broadly expressed in all major cell types, i.e., secretory cells, ciliated cells, and basal cells, as well as ionocytes. WNK2 was also abundantly expressed in secretory cells and ionocytes. In contrast to WNK1, WNK2 was rarely detected in ciliated or basal cells. The remaining WNK isoforms, WNK3 and WNK4, were either not expressed, or expressed at a very low level. We also studied the expression of the two main downstream kinases, i.e., STK39 which encodes Ste20/SPS1-related proline-alanine-rich protein kinase (SPAK), and OXSR1 which encodes oxidative stress responsive 1 kinase (OSR1). Both genes were broadly expressed, and abundantly detected in secretory cells as well as ionocytes. We did not detect any differences in expression of these genes in CF versus non-CF epithelia (FIGS. 8A-8F).

To reveal WNK protein expression, we immunolabeled CF epithelia for WNK1 and WNK2 (FIG. 1B). In agreement with scRNA-seq results, we detected WNK1 in ciliated as well as non-ciliated cells, and WNK2 predominantly in non-ciliated cells. Further immunolocalization studies revealed WNK1 and WNK2 expression in secretory cells (labeled with anti-CC10 antibody) as well as ionocytes (labeled with anti-BSND antibody) (FIGS. 1C and 1D). Overall, these studies identified two WNK kinases in airway cells that secrete anions.

WNK inhibition increases CF pH_{ASL}. Several HCO₃⁻ and H⁺ transport mechanisms integrate to determine pH_{ASL}, and pH_{ASL} determines host defense (13, 19). To begin to understand the role of WNK kinases in regulating pH_{ASL}, we used pharmacologic WNK inhibition. WNK463 is a selective, ATP-competitive, pan-WNK kinase inhibitor; and has recently emerged as a useful tool for studying ion transport physiology (50-53). We exposed airway epithelia to either vehicle or WNK463 for 2 hours, and measured pH_{ASL} in an environment containing 25 mM HCO₃⁻ and 5% CO₂. In primary cultures of both CF and non-CF epithelia, WNK463 increased pH_{ASL} (FIGS. 2A and 2B). As an additional test, we also studied NuLi-1 (WT/WT) and CuFi-4 (G551D/ΔF508) epithelia and elicited similar responses to WNK463 (FIGS. 2C and 2D). Importantly, alkalinization in CF epithelia indicated that the WNK463⁻induced response did not require CFTR. When studied in a nominally HCO₃⁻/CO₂⁻free environment, the pH_{ASL} response disappeared (FIG. 2E). This result suggested that WNK463 increased CF pH_{ASL} by increasing HCO₃⁻ secretion, and not by decreasing H⁺ secretion. Next, we asked whether this response was time-dependent. Two hours of exposure increased pH_{ASL}, and continued exposure up to 24 hours did not further alkalinize ASL (FIG. 2F).

WNK kinases modulate membrane transporters either directly, or indirectly through their native substrates, SPAK and OSR1 (54) (FIG. 2G). In scRNA-seq data, cell types expressing WNK1 and WNK2 also expressed genes encoding SPAK and OSR1. To test the latter's involvement in controlling pH_{ASL}, we treated CF epithelia with rafoxanide, an allosteric SPAK/OSR1 inhibitor (55). Similar to WNK463, rafoxanide applied for 2 hours also increased CF pH_{ASL} (FIG. 2H). Taken together, these responses suggested that CF pH_{ASL} is controlled by upstream as well as downstream kinases in the canonical WNK/SPAK/OSR1 signaling pathway.

WNK463 enhances CF host defenses. Previous studies showed that alkalinizing CF ASL improves respiratory host defenses (12, 19-21). Because WNK463 increased pH_{ASL}, we tested its impact. Defective mucus transport is a key feature of CF (56). In primary cultures of differentiated CF epithelia, WNK463 decreased ASL viscosity (FIG. 3A), consistent with previous studies showing that increasing pH_{ASL} decreases viscosity. In addition, WNK463 did not alter the rate of apical liquid absorption (FIG. 3B), suggesting that a change in apical fluid volume was not involved. WNK463 also increased ciliary beat frequency, albeit modestly (FIG. 3C). Both decrease in viscosity and increase in ciliary beat frequency would improve CF mucus transport. Previous studies also indicated that CF ASL has reduced antibacterial activity (13, 57). WNK463 increased ASL-mediated S. aureus killing in primary CF epithelia (FIG. 3D). Overall, these results suggested that targeting WNK kinase signaling may at least partially rescue CF host defense defects.

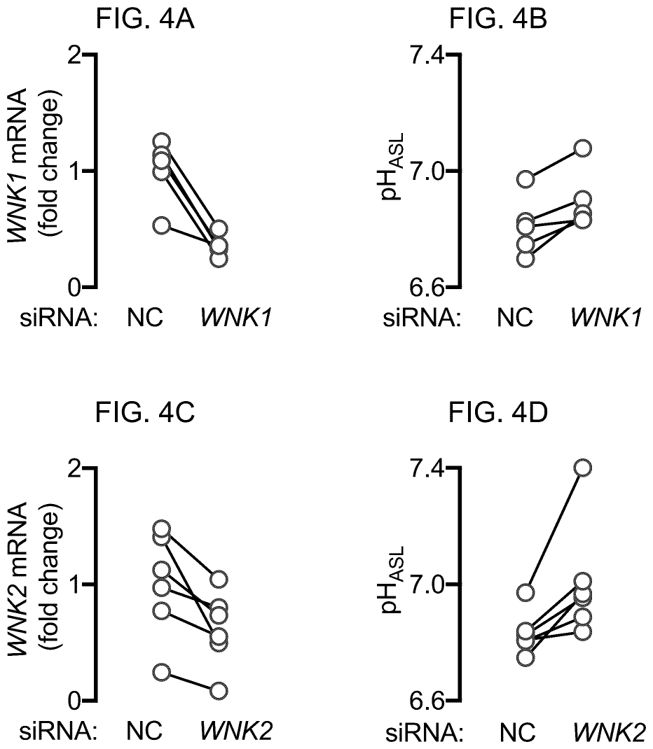

Either WNK1- or WNK2⁻knockdown increases CF pH_{ASL}. WNK463 is a pan-WNK kinase inhibitor (50). Because airway epithelia expressed two WNK kinases, we asked whether WNK1 or WNK2 controlled CF pH_{ASL}. To test, we performed siRNA-mediated gene knockdown. Reducing either WNK1 or WNK2 expression increased CF pH_{ASL} (FIGS. 4A-4D). This result suggested that both isoforms, WNK1 and WNK2, participate in regulating CF pH_{ASL}.

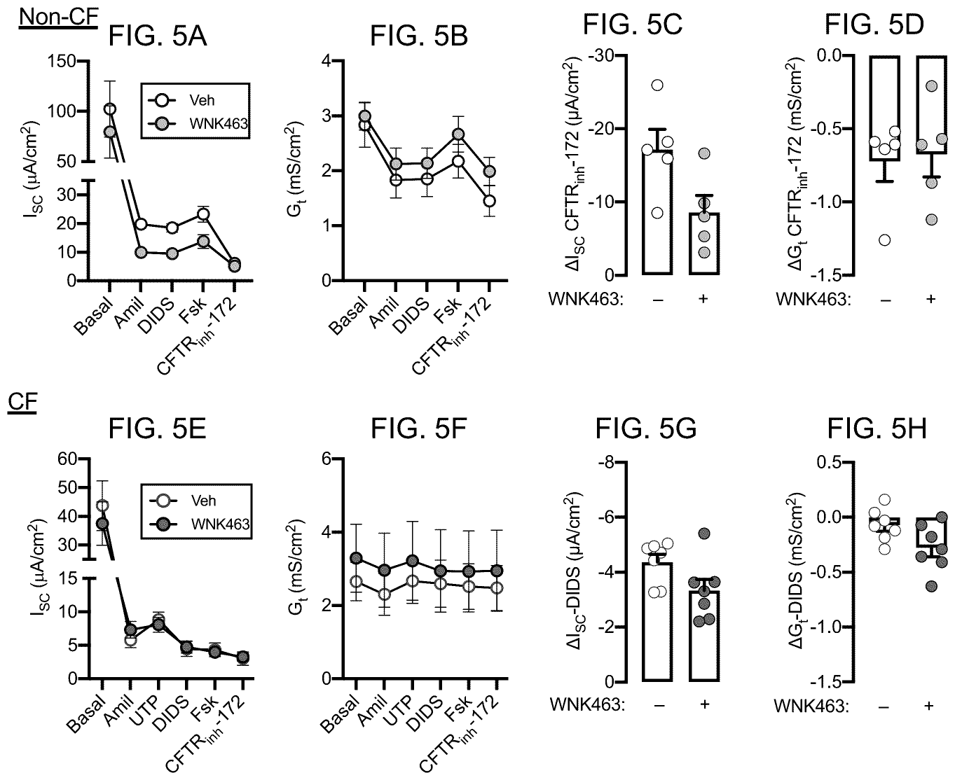

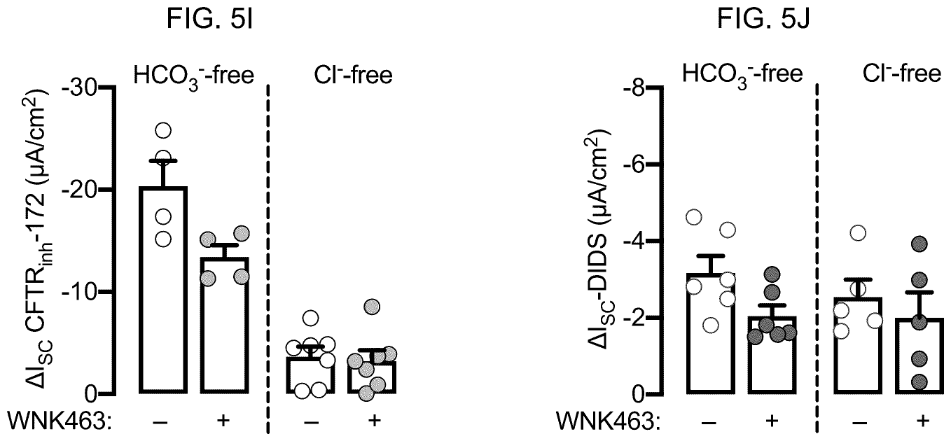

WNK463 reduces electrogenic Cl⁻ secretion. CFTR is the main route for anion exit across the apical membrane of airway epithelia. Whether WNK inhibition alters CFTR activity in airway epithelia is not well-established. To test, we exposed non-CF epithelia to WNK463 for 2 hours, and assayed in Ussing chambers containing symmetric Krebs solution (118 mM Cl⁻ and 25 mM HCO₃⁻, gassed with 5% CO₂). After clamping transepithelial voltage, we recorded I_{SC} and Gₜ, and elicited responses to selective channel inhibitors or activators (FIGS. 5A and 5B). We added amiloride followed by DIDS to abolish ENaC-mediated Na⁺ absorption and Ca²⁺-activated Cl⁻ channel (CaCC)-mediated anion secretion, respectively. Next, we added forskolin to increase cellular cAMP and thereby phosphorylate and activate CFTR channels. We concluded with CFTRᵢₙₕ-172, an inhibitor of CFTR. We assessed the response to CFTRᵢₙₕ-172 and used it to estimate CFTR channel activity. WNK463 reduced ΔI_{SC}-CFTR by ˜ 50% (FIG. 5C). However, ΔGₜ-CFTR remained unchanged (FIG. 5D). This result suggested that WNK463 decreased CFTR-mediated anion transport but did not alter CFTR channel activity at the apical membrane.

CF epithelia lack functional CFTR channels, but express CaCC. Accordingly, we studied the effect of WNK463 on CaCC-mediated anion transport in CF epithelia (FIGS. 5, E and F). After blocking ENaC with amiloride, we added UTP, a P2Y2 purinergic receptor agonist that increases cytosolic [Ca²⁺] and thus activates CaCC. Next, we added DIDS, a non-specific CaCC inhibitor, and recorded the change in I_{SC} and Gₜ. WNK463 decreased DIDS-sensitive ΔI_{SC} but slightly increased DIDS-sensitive ΔGₜ (FIGS. 5G and 5H). Together, these findings suggested that inhibiting WNK kinases reduces anion secretion, but the effect is not on apical anion channels.

To separate the effects of WNK inhibition on Cl⁻ versus HCO₃⁻ transport, we repeated the studies in single anion solutions. In symmetric HCO₃⁻-free solution, WNK463 reduced ΔI_{SC}-CFTR (FIG. 5I); however, in Cl⁻-free solution, ΔI_{SC}-CFTR remained unchanged. Similar results were obtained for the DIDS-sensitive ΔI_{SC} in CF epithelia (FIG. 5J). These data suggested that WNK inhibition reduces electrogenic Cl⁻ secretion but does not alter electrogenic HCO₃⁻ secretion.

Reducing basolateral Cl⁻-entry increases CF pH_{ASL}. Transcellular Cl secretion involves the movement of Cl⁻ across the apical and the basolateral membranes in series. Because studies of electrically conductive anion transport showed reduced Cl⁻ secretion without major effects at the apical membrane, we asked whether a change at the basolateral membrane was involved. The loop-sensitive Na⁺—K⁺-2Cl⁻ (NKCC) cotransporter is the main route for Cl⁻ entry across the basolateral membrane, and WNK kinases are known to increase NKCC activity in renal epithelia (58, 59). To further investigate the effect of WNK463 on this transport mechanism, we studied non-CF and CF epithelia in Ussing chambers. After blocking ENaC with amiloride, we added either forskolin to activate CFTR in non-CF epithelia, or UTP to activate CaCC in CF epithelia. To estimate the contribution of NKCC1, we added basolateral bumetanide, and measured ΔI_{SC}. WNK463 reduced bumetanide-sensitive I_{SC} in both non-CF and CF epithelia (FIGS. 6A and 6B). This result pointed to the involvement of WNK kinases in controlling basolateral Cl⁻uptake through a bumetanide-sensitive mechanism.

Previous studies in airway epithelia showed that bumetanide decreases intracellular [Cl⁻] (60-62). This led us to hypothesize that lowering intracellular [Cl⁻] might also increase CF pH_{ASL}. To test, we performed two experiments: 1) We tested the effect of NKCC1 inhibition in CF epithelia. Exposure to bumetanide increased CF pH_{ASL} (FIG. 6C, left panel). 2) We measured pH_{ASL} in a Cl⁻free environment. Similar to bumetanide, the removal of basolateral Cl⁻ also increased CF pH_{ASL} (FIG. 6C, right panel). Because WNK463 reduced bumetanide-sensitive I_{SC}, and lowering NKCC1 activity or intracellular [Cl⁻] increased pH_{ASL}, we considered whether intracellular [Cl⁻] was involved in the response evoked by WNK463. When introduced in the absence of Cl⁻, WNK463 failed to alkalinize CF ASL, thus indicating that the WNK463⁻ elicited pH_{ASL} response was Cl⁻-dependent (FIG. 6D).

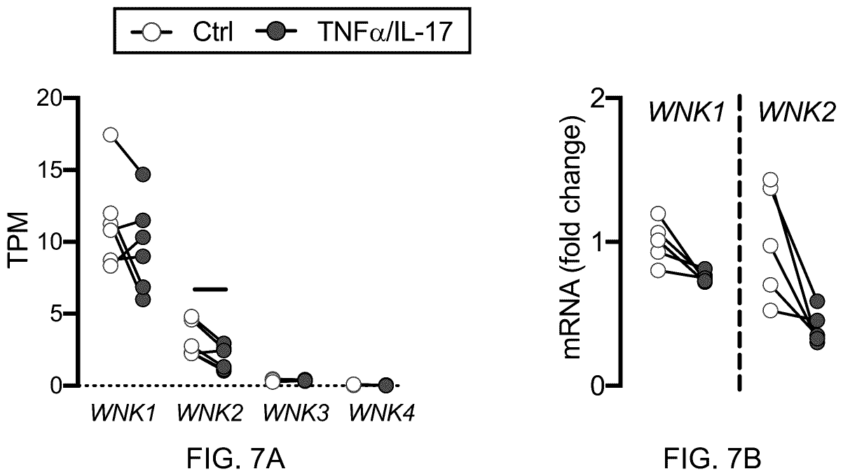

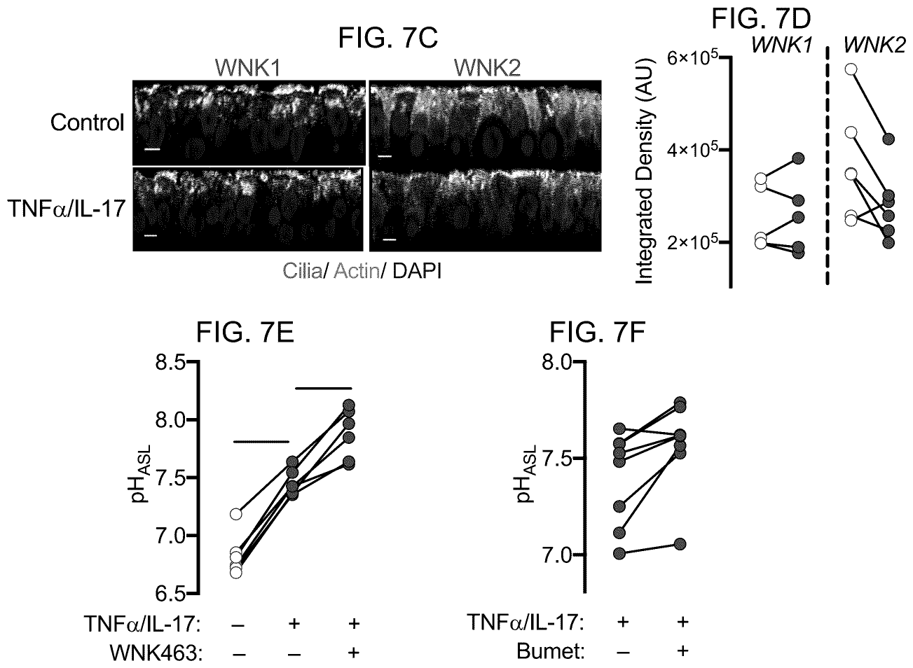

WNK463 further increases pH_{ASL} in TNFα/IL-17⁻treated CF epithelia. Airway inflammation is ubiquitous in CF individuals after the first few weeks of life (63-65). The CF airway inflammation is characteristically neutrophil-predominant, may develop in the absence of infection, and is further exacerbated by infection and colonization. Two CF-relevant inflammatory cytokines, TNFα and IL-17, drive neutrophilic inflammation (66-69). In previous work, combined TNFα/IL-17 increased HCO₃⁻ secretion and CF pH_{ASL} by increasing pendrin expression (43, 70). We asked if TNFα/IL-17⁻induced alkalinization was also accompanied by altered expression of WNK kinases. In gene expression studies, TNFα/IL-17 modestly reduced WNK1, and markedly reduced WNK2 expression (FIGS. 7A and 7B). In immunocytochemistry studies, TNFα/IL-17 decreased WNK2 detection, but WNK1 remained unchanged (FIGS. 7C and 7D). This led us to hypothesize that residual WNK kinases might continue to regulate HCO₃⁻ secretion in cytokine-treated epithelia. Accordingly, exposure to WNK463 further increased pH_{ASL} in CF epithelia treated with TNFα/IL-17 (FIG. 7E). Since WNK463 decreased Cl⁻ secretion, and reducing basolateral Cl⁻ entry increased CF pH_{ASL}, we predicted a similar response to lowering basolateral Cl⁻ entry in cytokine-treated CF epithelia. Exposure to bumetanide further alkalinized ASL in TNFα/IL-17⁻treated CF epithelia (FIG. 7F). This result suggested that TNFα/IL-17 shifted apical anion secretion in favor of HCO₃⁻ over Cl⁻, and lowering basolateral Cl⁻ entry further augmented this response.

### DISCUSSION

Our transcript and immunocytochemistry data for WNK1 and WNK2, and their substrates STK39 and OXSR1, indicated that these kinases are expressed in secretory cells and ionocytes, the main airway epithelial cells that secrete anions. Consistent with that localization, pharmacologically inhibiting WNK kinases, SPAK/OSR1 kinases, and knocking down WNK1 and WNK2 transcripts increased pH_{ASL}. These results thus identified an important role for WNK kinases in regulating HCO₃⁻ secretion across airway epithelia.

Electrophysiological studies indicated that inhibiting WNK kinases decreased the Cl⁻-mediated, but not HCO₃⁻-mediated current. Moreover, ASL alkalinization persisted in the absence of CFTR activity in CF epithelia. A clue to a potential mechanism came with the finding that inhibiting WNK kinases largely eliminated the inhibitory effect of basolateral bumetanide on I_{SC}. Bumetanide inhibits NKCC1, the major pathway for Cl⁻ entry into the cell, and thereby reduces the intracellular [Cl⁻] (60-62). Further evidence implicating intracellular [Cl⁻] came from studies showing that adding bumetanide alone or removing Cl⁻ from the medium also alkalinized ASL in CF epithelia.

These results suggest that WNK kinases may play a key role in determining the balance between Cl⁻ secretion and HCO₃⁻ secretion across airway epithelia. Inhibiting WNK kinases decreased NKCC1 activity, which decreased Cl⁻ secretion, increased HCO₃⁻ secretion, and increased pH_{ASL}. The inference that inhibiting WNK reduces NKCC1 activity is supported by the finding that bumetanide also increased pH_{ASL} and previous reports that WNK kinases increase NKCC activity in non-airway epithelia (58, 59). However, the mechanism that increases HCO₃⁻ secretion is uncertain. One possibility is that WNK inhibition reduces the intracellular [Cl⁻] thereby increasing the driving force for Cl⁻/HCO₃ exchange at the apical membrane and hence HCO₃⁻ secretion. Finding that bumetanide replicates the effect of WNK inhibition on pH_{ASL} is consistent with this hypothesis. However, Cl⁻-free bathing solution also induced HCO₃⁻ secretion despite the fact that reduced [Cl⁻] would initially and transiently drive Cl⁻/HCO₃ exchange in the opposite direction, and over the 2-hour time course of the experiment, cellular Cl⁻ would be largely depleted. Thus, we favor an alternative explanation that intracellular Cl⁻ is a signaling molecule that regulates membrane transport (71).

Intracellular [Cl⁻] regulation of HCO₃; secretion has been reported previously. A Cl⁻-sensing motif has been identified in some HCO₃⁻ transporters and other proteins (35, 72). Low intracellular [Cl⁻] was shown to increase IP3 receptor binding protein released with IP3 (IRBIT)-stimulated NBCel-B activity (72). Kim et al. showed that low intracellular [Cl⁻] enabled structural association between WNK1 and CFTR, and increased CFTR HCO₃⁻ channel activity (35). Notably, this effect did not depend on WNK1 kinase activity. Yamaguchi et al. developed a computational model of guinea pig pancreatic duct HCO₃⁻ secretion (73). In this model, maximal HCO₃⁻ secretion did not depend on an increase in CFTR P_{HCO3}/P_{Cl} or a change in SLC26 Cl⁻/HCO₃⁻ exchange stoichiometry, but instead depended on suppression of basolateral Cl⁻ uptake. The addition of NKCC1, normally missing from guinea pig pancreatic ducts, increased intracellular [Cl⁻] and reduced secreted [HCO₃⁻]. Our results also support intracellular [Cl]-dependent regulation of HCO₃⁻ secretion. It will be important for future studies to establish underlying molecular mechanisms in primary airway epithelia.

We previously reported that combined TNFα/IL-17 increased production of pendrin, an apical Cl⁻/HCO₃⁻ exchanger, and alkalinized CF ASL (43, 70). Here, we show that TNFα/IL-17 also reduced WNK2 expression. Moreover, inhibiting the residual WNK kinase activity with WNK463 further increased pH_{ASL}, and basolateral bumetanide mimicked the effect of WNK inhibition. These pro-inflammatory cytokines may thus induce HCO₃⁻ secretion and increase pH_{ASL} by at least two mechanisms, increasing pendrin expression and reducing WNK2 expression. Whether WNK kinases regulate apical expression or activity of pendrin in cytokine-treated airway epithelia remains to be determined.

This study has several advantages. First, we studied primary cultures of differentiated human airway epithelia from both CF and non-CF genotypes. Second, to account for biological variability, we included epithelia from multiple human donors. Third, we measured pH_{ASL} under thin-film conditions without adding additional apical fluid. Fourth, in testing our hypothesis, we used a combination of pharmacologic, transcriptomic, gene silencing, protein immunolabeling, and electrophysiologic approaches. Fifth, we also studied responses in established human airway epithelial cell lines, NuLi-1 and CuFi-4 (38). Finding that WNK signaling is active in these epithelia enables their use as models for studying WNK signaling.

This study also has limitations. First, we used human airway epithelia as an in vitro model; and assessing WNK kinase inhibition in vivo may be of value. However, interpretation of in vivo effects may be complicated by the fact that WNK kinases are expressed broadly in epithelial and non-epithelial cells (44, 45). Second, we did not identify the transporter directly responsible for apical HCO₃⁻ exit. RNA-seq studies show that CF airway epithelia express several non-CFTR HCO₃⁻ transporters including CaCC and SLC26 family members (43, 74), and WNK inhibition may affect more than one simultaneously.

The results have implications for CF airways. First, previous studies have shown that the abnormally acidic pH_{ASL} observed in CF newborns increases with time and inflammation (75, 76), although not to levels observed in non-CF epithelia studied under comparable conditions (43). As indicated above, our current data together with previous results suggest complex regulatory mechanisms are responsible. Second, loop diuretics, which inhibit NKCC, are commonly used to treat heart failure and fluid overload states (77). Yet, to our knowledge, these agents have not been shown to cause adverse airway phenotypes. This study suggests that HCO₃⁻ secretion may compensate for any decrease in loop-sensitive Cl⁻ secretion and preserve, if not augment, host defenses (78). Third, by enhancing respiratory host defense, WNK inhibition might be a potential therapeutic target in CF. Although WNK463 produced adverse effects in a rat model of hypertension (50), it is a non-selective WNK kinase inhibitor. Interestingly, WNK2 has a more restricted tissue expression than the ubiquitous WNK1, is detected in airway epithelial cell types relevant for anion secretion, and its knockdown alkalinizes CF ASL. Thus, selective WNK2 inhibitors, inhibitors of downstream SPAK/OSR1 kinases, or inhibitors restricted to the airways might be pursued as potential CF therapeutics.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

### REFERENCES

1. Quinton P M. Physiological basis of cystic fibrosis: a historical perspective. Physiol Rev. 1999; 79(1 Suppl):S3⁻S22.
2. Ratjen F, Bell S C, Rowe S M, Goss C H, Quittner A L, Bush A. Cystic fibrosis. Nat Rev Dis Primers. 2015; 1:15010.
3. Stoltz D A, Meyerholz D K, Welsh M J. Origins of cystic fibrosis lung disease. N Engl J Med. 2015; 372(16):1574-5.
4. Poulsen J H, Fischer H, Illek B, Machen T E. Bicarbonate conductance and pH regulatory capability of cystic fibrosis transmembrane conductance regulator. Proc Natl Acad Sci USA. 1994; 91(12):5340-4.
5. Tang L, Fatehi M, Linsdell P. Mechanism of direct bicarbonate transport by the CFTR anion channel. J Cyst Fibros. 2009; 8(2):115-21.
6. Sheppard D N, Welsh M J. Structure and function of the CFTR chloride channel. Physiol Rev. 1999; 79(1 Suppl):523-45.
7. Widdicombe J H. Airway epithelium. San Rafael, Calif: Morgan & Claypool Life Sciences; 2013.
8. Haq I J, Gray M A, Garnett J P, Ward C, Brodlie M. Airway surface liquid homeostasis in cystic fibrosis: pathophysiology and therapeutic targets. Thorax. 2016; 71(3):284-7.
9. Verkman A S, Song Y, Thiagarajah J R. Role of airway surface liquid and submucosal glands in cystic fibrosis lung disease. Am J Physiol Cell Physiol. 2003; 284(1):C2-15.
10. Gustafsson J K, Ermund A, Ambort D, Johansson M E, Nilsson H E, Thorell K, et al. Bicarbonate and functional CFTR channel are required for proper mucin secretion and link cystic fibrosis with its mucus phenotype. J Exp Med. 2012; 209(7):1263-72.
11. Zajac M, Dreano E, Edwards A, Planelles G, Sermet-Gaudelus I. Airway Surface Liquid pH Regulation in Airway Epithelium Current Understandings and Gaps in Knowledge. Int J Mol Sci. 2021; 22(7).
12. Tang X X, Ostedgaard L S, Hoegger M J, Moninger T O, Karp P H, McMenimen J D, et al. Acidic pH increases airway surface liquid viscosity in cystic fibrosis. J Clin Invest. 2016; 126(3):879-91.
13. Pezzulo A A, Tang X X, Hoegger M J, Abou Alaiwa M H, Ramachandran S, Moninger T O, et al. Reduced airway surface pH impairs bacterial killing in the porcine cystic fibrosis lung. Nature. 2012; 487(7405):109-13.
14. Abou Alaiwa M H, Reznikov L R, Gansemer N D, Sheets K A, Horswill A R, Stoltz D A, et al. pH modulates the activity and synergism of the airway surface liquid antimicrobials beta-defensin-3 and LL-37. Proc Natl Acad Sci USA. 2014; 111(52):18703-8.
15. Simonin J, Bille E, Crambert G, Noel S, Dreano E, Edwards A, et al. Airway surface liquid acidification initiates host defense abnormalities in Cystic Fibrosis. Sci Rep. 2019; 9(1):6516.
16. Quinton P M. Cystic fibrosis: impaired bicarbonate secretion and mucoviscidosis. Lancet. 2008; 372(9636):415-7.
17. Nakayama K, Jia Y X, Hirai H, Shinkawa M, Yamaya M, Sekizawa K, et al. Acid stimulation reduces bactericidal activity of surface liquid in cultured human airway epithelial cells. Am J Respir Cell Mol Biol. 2002; 26(1):105-13.
18. Birket S E, Davis J M, Fernandez C M, Tuggle K L, Oden A M, Chu K K, et al. Development of an airway mucus defect in the cystic fibrosis rat. JCI Insight. 2018; 3(1).
19. Shah V S, Meyerholz D K, Tang X X, Reznikov L, Abou Alaiwa M, Ernst S E, et al. Airway acidification initiates host defense abnormalities in cystic fibrosis mice. Science. 2016; 351(6272):503-7.
20. Abou Alaiwa M H, Launspach J L, Sheets K A, Rivera J A, Gansemer N D, Taft P J, et al. Repurposing tromethamine as inhaled therapy to treat C F airway disease. JCI Insight. 2016; 1(8).
21. Muraglia K A, Chorghade R S, Kim B R, Tang X X, Shah V S, Grillo A S, et al. Small-molecule ion channels increase host defences in cystic fibrosis airway epithelia. Nature. 2019; 567(7748):405-8.
22. Sindic A, Sussman C R, Romero M F. Primers on molecular pathways: bicarbonate transport by the pancreas. Pancreatology. 2010; 10(6):660-3.
23. Fischer H, Widdicombe J H. Mechanisms of acid and base secretion by the airway epithelium. J Membr Biol. 2006; 211(3):139-50.
24. Cordat E, Casey J R. Bicarbonate transport in cell physiology and disease. Biochem J. 2009; 417(2):423-39.
25. Alka K, Casey J R. Bicarbonate transport in health and disease. IUBMB Life. 2014; 66(9):596-615.
26. Lee M G, Ohana E, Park H W, Yang D, Muallem S. Molecular mechanism of pancreatic and salivary gland fluid and HCO₃ secretion. Physiol Rev. 2012; 92(1):39-74.
27. Shah V S, Chivukula R R, Lin B, Waghray A, Rajagopal J. Cystic Fibrosis and the Cells of the Airway Epithelium: What Are Ionocytes and What Do They Do? Annu Rev Pathol. 2022; 17:23-46.
28. Kunzelmann K, Schreiber R, Hadom H B. Bicarbonate in cystic fibrosis. J Cyst Fibros. 2017; 16(6):653-62.
29. Li H, Salomon J J, Sheppard D N, Mall M A, Galietta U. Bypassing CFTR dysfunction in cystic fibrosis with alternative pathways for anion transport. Curr Opin Pharmacol. 2017; 34:91-7.
30. Shekarabi M, Zhang J, Khanna A R, Ellison D H, Delpire E, Kahle K T. WNK Kinase Signaling in Ion Homeostasis and Human Disease. Cell Metab. 2017; 25(2):285-99.
31. Park S, Hong J H, Ohana E, Muallem S. The WNK/SPAK and IRBIT/PP1 pathways in epithelial fluid and electrolyte transport. Physiology (Bethesda). 2012; 27(5):291-9.
32. Angyal D, Bijvelds M J C, Bruno M J, Peppelenbosch M P, de Jonge H R. Bicarbonate Transport in Cystic Fibrosis and Pancreatitis. Cells. 2021; 11(1).
33. Yang D, Li Q, So I, Huang C L, Ando H, Mizutani A, et al. IRBIT governs epithelial secretion in mice by antagonizing the WNK/SPAK kinase pathway. J Clin Invest. 2011; 121(3):956-65.
34. Park H W, Nam J H, Kim J Y, Namkung W, Yoon J S, Lee J S, et al. Dynamic regulation of CFTR bicarbonate permeability by [Cl⁻]i and its role in pancreatic bicarbonate secretion. Gastroenterology. 2010; 139(2):620-31.
35. Kim Y, Jun I, Shin D H, Yoon J G, Piao H, Jung J, et al. Regulation of CFTR Bicarbonate Channel Activity by WNK1: Implications for Pancreatitis and CFTR-Related Disorders. Cell Mol Gastroenterol Hepatol. 2020; 9(1):79-103.
36. Dorwart M R, Shcheynikov N, Wang Y, Stippec S, Muallem S. SLC26A9 is a Cl(−) channel regulated by the WNK kinases. J Physiol. 2007; 584(Pt 1):333-45.
37. Karp P H, Moninger T O, Weber S P, Nesselhauf T S, Launspach J L, Zabner J, et al. An in vitro model of differentiated human airway epithelia. Methods for establishing primary cultures. Methods Mol Biol. 2002; 188:115-37.
38. Zabner J, Karp P, Seiler M, Phillips S L, Mitchell C J, Saavedra M, et al. Development of cystic fibrosis and noncystic fibrosis airway cell lines. Am J Physiol Lung Cell Mol Physiol. 2003; 284(5):L844-54.
39. Stuart T, Butler A, Hoffman P, Hafemeister C, Papalexi E, Mauck W M, 3rd, et al. Comprehensive Integration of Single-Cell Data. Cell. 2019; 177(7):1888-902 e21.
40. Zabner J, Smith J J, Karp P H, Widdicombe J H, Welsh M J. Loss of CFTR chloride channels alters salt absorption by cystic fibrosis airway epithelia in vitro. Mol Cell. 1998; 2(3):397-403.
41. Sisson J H, Stoner J A, Ammons B A, Wyatt T A. All-digital image capture and whole-field analysis of ciliary beat frequency. J Microsc. 2003; 211(Pt 2):103-11.
42. Ramachandran S, Krishnamurthy S, Jacobi A M, Wohlford-Lenane C, Behlke M A, Davidson B L, et al. Efficient delivery of RNA interference oligonucleotides to polarized airway epithelia in vitro. Am J Physiol Lung Cell Mol Physiol. 2013; 305(1):L23-32.
43. Rehman T, Karp P H, Tan P, Goodell B J, Pezzulo A A, Thurman A L, et al. Inflammatory cytokines TNF-alpha and IL-17 enhance the efficacy of cystic fibrosis transmembrane conductance regulator modulators. J Clin Invest. 2021; 131(16).
44. McCormick J A, Ellison D H. The WNKs: atypical protein kinases with pleiotropic actions. Physiol Rev. 2011; 91(1):177-219.
45. Consortium G T. Human genomics. The Genotype-Tissue Expression (GTEx) pilot analysis: multitissue gene regulation in humans. Science. 2015; 348(6235):648-60.
46. Plasschaert L W, Zilionis R, Choo-Wing R, Savova V, Knehr J, Roma G, et al. A single-cell atlas of the airway epithelium reveals the CFTR-rich pulmonary ionocyte. Nature. 2018; 560(7718):377-81.
47. Montoro D T, Haber A L, Biton M, Vinarsky V, Lin B, Birket S E, et al. A revised airway epithelial hierarchy includes CFTR-expressing ionocytes. Nature. 2018; 560(7718):319-24.
48. Okuda K, Dang H, Kobayashi Y, Carraro G, Nakano S, Chen G, et al. Secretory Cells Dominate Airway CFTR Expression and Function in Human Airway Superficial Epithelia. Am J Respir Crit Care Med. 2021; 203(10):1275-89.
49. Barbry P, Marcet B, Caballero I. Where Is the Cystic Fibrosis Transmembrane Conductance Regulator? Am J Respir Crit Care Med. 2021; 203(10):1214-6.
50. Yamada K, Park H M, Rigel D F, DiPetrillo K, Whalen E J, Anisowicz A, et al. Small-molecule WNK inhibition regulates cardiovascular and renal function. Nat Chem Biol. 2016; 12(11):896-8.
51. Lu D C, Hannemann A, Wadud R, Rees D C, Brewin J N, Low P S, et al. The role of WNK in modulation of KCl cotransport activity in red cells from normal individuals and patients with sickle cell anaemia. Pflugers Arch. 2019; 471(11-12):1539-49.
52. Mayes-Hopfinger L, Enache A, Xie J, Huang C L, Kochl R, Tybulewicz V L J, et al. Chloride sensing by WNK1 regulates NLRP3 inflammasome activation and pyroptosis. Nat Commun. 2021; 12(1):4546.
53. Zhang J, Deng X, Kahle K T. Leveraging unique structural characteristics of WNK kinases to achieve therapeutic inhibition. Sci Signal. 2016; 9(450):e3.
54. Alessi D R, Zhang J, Khanna A, Hochdorfer T, Shang Y, Kahle K T. The WNK-SPAK/OSRT pathway: master regulator of cation-chloride cotransporters. Sci Signal. 2014; 7(334):re3.
55. AlAmri M A, Kadri H, Alderwick L J, Simpkins N S, Mehellou Y. Rafoxanide and Closantel Inhibit SPAK and OSR1 Kinases by Binding to a Highly Conserved Allosteric Site on Their C-terminal Domains. ChemMedChem. 2017; 12(9):639-45.
56. Wine J J, Hansson G C, Konig P, Joo N S, Ermund A, Pieper M. Progress in understanding mucus abnormalities in cystic fibrosis airways. J Cyst Fibros. 2018; 17(2S):535-S9.
57. Smith J J, Travis S M, Greenberg E P, Welsh M J. Cystic fibrosis airway epithelia fail to kill bacteria because of abnormal airway surface fluid. Cell. 1996; 85(2):229-36.
58. Kahle K T, Rinehart J, Lifton R P. Phosphoregulation of the Na—K-2C1 and K—Cl cotransporters by the WNK kinases. Biochim Biophys Acta. 2010; 1802(12):1150-8.
59. Huang C L, Yang S S, Lin S H. Mechanism of regulation of renal ion transport by WNK kinases. Curr Opin Nephrol Hypertens. 2008; 17(5):519-25.
60. Welsh M J. Intracellular chloride activities in canine tracheal epithelium. Direct evidence for sodium-coupled intracellular chloride accumulation in a chloride-secreting epithelium. J Clin Invest. 1983; 71(5):1392-401.
61. Willumsen N J, Davis C W, Boucher R C. Intracellular Cl— activity and cellular Cl— pathways in cultured human airway epithelium. Am J Physiol. 1989; 256(5 Pt 1):C1033-44.
62. Willumsen N J, Davis C W, Boucher R C. Cellular Cl⁻ transport in cultured cystic fibrosis airway epithelium. Am J Physiol. 1989; 256(5 Pt 1):C1045-53.
63. Sly P D, Brennan S, Gangell C, de Klerk N, Murray C, Mott L, et al. Lung disease at diagnosis in infants with cystic fibrosis detected by newborn screening. Am J Respir Crit Care Med. 2009; 180(2):146-52.
64. Schultz A, Stick S. Early pulmonary inflammation and lung damage in children with cystic fibrosis. Respirology. 2015; 20(4):569-78.
65. Sly P D, Gangell C L, Chen L, Ware R S, Ranganathan S, Mott L S, et al. Risk factors for bronchiectasis in children with cystic fibrosis. N Engl J Med. 2013; 368(21):1963⁻ 70.
66. Lukacs N W, Strieter R M, Chensue S W, Widmer M, Kunkel S L. TNF-alpha mediates recruitment of neutrophils and eosinophils during airway inflammation. J Immunol. 1995; 154(10):5411-7.
67. Karpati F, Hjelte F L, Wretlind B. TNF-alpha and IL-8 in consecutive sputum samples from cystic fibrosis patients during antibiotic treatment. Scand J Infect Dis. 2000; 32(1):75-9.
68. McAllister F, Henry A, Kreindler J L, Dubin P J, Ulrich L, Steele C, et al. Role of IL-17A, IL-17F, and the IL-17 receptor in regulating growth-related oncogene-alpha and granulocyte colony-stimulating factor in bronchial epithelium: implications for airway inflammation in cystic fibrosis. J Immunol. 2005; 175(1):404-12.
69. Tan H L, Regamey N, Brown S, Bush A, Lloyd C M, Davies J C. The Th17 pathway in cystic fibrosis lung disease. Am J Respir Crit Care Med. 2011; 184(2):252-8.
70. Rehman T, Thomell I M, Pezzulo A A, Thurman A L, Romano Ibarra G S, Karp P H, et al. TNFalpha and IL-17 alkalinize airway surface liquid through CFTR and pendrin. Am J Physiol Cell Physiol. 2020; 319(2):C331⁻C44.
71. Luscher B P, Vachel L, Ohana E, Muallem S. Cl(−) as a bona fide signaling ion. Am J Physiol Cell Physiol. 2020; 318(1):C125⁻C36.
72. Shcheynikov N, Son A, Hong J H, Yamazaki O, Ohana E, Kurtz I, et al. Intracellular Cl⁻ as a signaling ion that potently regulates Na⁺/HCO₃⁻ transporters. Proc Natl Acad Sci USA. 2015; 112(3):E329-37.
73. Yamaguchi M, Steward M C, Smallbone K, Sohma Y, Yamamoto A, Ko S B, et al. Bicarbonate-rich fluid secretion predicted by a computational model of guinea-pig pancreatic duct epithelium. J Physiol. 2017; 595(6):1947-72.
74. Carraro G, Langerman J, Sabri S, Lorenzana Z, Purkayastha A, Zhang G, et al. Transcriptional analysis of cystic fibrosis airways at single-cell resolution reveals altered epithelial cell states and composition. Nat Med. 2021; 27(5):806-14.
75. Schultz A, Puvvadi R, Borisov S M, Shaw N C, Klimant I, Berry L J, et al. Airway surface liquid pH is not acidic in children with cystic fibrosis. Nat Commun. 2017; 8(1):1409.
76. Abou Alaiwa M H, Beer A M, Pezzulo A A, Launspach J L, Horan R A, Stoltz D A, et al. Neonates with cystic fibrosis have a reduced nasal liquid pH; a small pilot study. J Cyst Fibros. 2014; 13(4):373-7.
77. Oh S W, Han S Y. Loop Diuretics in Clinical Practice. Electrolyte Blood Press. 2015; 13(1):17-21.
78. Grubb B R, Pace A J, Lee E, Koller B H, Boucher R C. Alterations in airway ion transport in NKCC1⁻deficient mice. Am J Physiol Cell Physiol. 2001; 281(2):C615-23.

1. A method of treating cystic fibrosis in a subject in need thereof, the method comprising: administering to the subject an effective amount of a WNK kinase pathway inhibitor to treat the cystic fibrosis.

2. A method of treating one or more signs or symptoms of cystic fibrosis in a subject in need thereof, the method comprising: administering to the subject an effective amount of a WNK kinase pathway inhibitor to the subject to treat the one or more signs or symptoms of cystic fibrosis.

3. The method of claim 2, wherein the one or more signs or symptoms of cystic fibrosis are selected from the group consisting of: persistent cough, wheezing, exercise intolerance, and repeated lung infections.

4. A method of increasing the pH of airway surface liquid (ASL) in a subject in need thereof, the method comprising: administering to the subject an effective amount of a WNK kinase pathway inhibitor to increase the pH of the ASL in the subject.

5. A method of enhancing respiratory defense in a subject in need thereof, the method comprising: administering to the subject an effective amount of a WNK kinase pathway inhibitor to enhance respiratory defense in the subject.

6. The method of any one of claims 1-5, wherein administering the WNK kinase pathway inhibitor causes increased secretion of HCO₃⁻ ions in the airway surface liquid of the subject.

7. The method of any one of claims 1-6, wherein the subject has a mutation in at least one allele of cystic fibrosis transmembrane conductance regulator (CFTR) gene.

8. The method of claim 7, wherein the mutation is F508^{del}.

9. The method of claim 7, wherein the mutation is not F508^{del}.

10. The method of claim 7, wherein the mutation is selected from a mutation, or combination of mutations, listed in Table 1.

11. The method of any one of claims 1-10, wherein the WNK kinase pathway inhibitor is selected from a WN1 inhibitor, a WNK2 inhibitor, a SPAK kinase inhibitor, or an OSR1 kinase inhibitor.

12. The method of any one of claims 1-11, wherein the WNK kinase pathway inhibitor inhibits WNR1 activity and/or WNK2 activity in airway epithelial cells in the subject.

13. The method of any one of claims 1-12, wherein the WNK kinase pathway inhibitor selectively inhibits WNK2.

14. The method of any one of claims 1-13, wherein the WNK kinase pathway inhibitor comprises a small molecule.

15. The method of any one of claims 1-14, wherein the WNK kinase pathway inhibitor comprises WNK463.

16. The method of any one of claims 1-15, wherein the method reduces Na—K-2C1 cotransporter-1 (NKCC1) activity.

17. The method of any one of claims 1-16, wherein the WNK kinase pathway inhibitor is administered intrapulmonarily.

18. A method of testing a compound of interest for use as a treatment for cystic fibrosis, comprising:
(a) culturing cells in the presence and absence of the compound of interest;
(b) culturing control cells in the presence and absence of a WNK kinase pathway inhibitor;
(c) detecting one or more parameters related to lung airway function in the cultured cells of step (a) and the cultured cells of step (b);
(d) generating a test index by calculating the change in the one or more parameters between the cultured cells of step (a) in the presence and absence of the compound of interest; and
(e) generating a control index by calculating the change in the one or more parameters between the cultured cells of step (b) in the presence and absence of the WNK kinase pathway inhibitor;
wherein if the value of the test index is equal to, or improved, as compared to the value of the control index, then the compound of interest is of use for the treatment of cystic fibrosis.

19. The method of claim 18, further comprising treating cystic fibrosis in a subject in need thereof by administering to the subject an effective amount of the compound of interest to treat cystic fibrosis.

20. The method of any one of claims 18 or 19, wherein the cells comprise differentiated primary human airway epithelial cells.

21. The method of any one of claims 18-20, wherein the cells comprise NuLi-1 or CuFi-4 cells.

22. The method of any one of claims 18-21, wherein the cells have a mutation in at least one allele of cystic fibrosis transmembrane conductance regulator (CFTR) gene.

23. The method of claim 22, wherein the mutation is F508^{del}.

24. The method of claim 22, wherein the mutation is not F508^{del}.

25. The method of claim 22, wherein the mutation is selected from a mutation or combination of mutations listed in Table 1.

26. The method of any one of claims 18-25, wherein the WNK kinase pathway inhibitor is selected from a WNK1 inhibitor, a WNK2 inhibitor, a SPAK kinase inhibitor, or an OSR1 kinase inhibitor.

27. The method of any one of claims 18-26, wherein the WNK kinase pathway inhibitor comprises a small molecule.

28. The method of any one of claims 18-27, wherein the WNK kinase pathway inhibitor comprises WNK463.

29. The method of any one of claims 18-28, wherein the WNK kinase pathway inhibitor selectively inhibits WNK2.

30. The method of any one of claims 18-29, wherein the WNK kinase pathway inhibitor reduces Na—K-2Cl cotransporter-1 (NKCC1) activity.

31. The method of any one of claims 18-30, wherein the one or more parameters are selected from pH, HCO₃⁻ secretion, Cl⁻ secretion, mucus viscosity, bacterial colonization, and electrical conductance across the membrane of the cells.


SEQUENCE LISTING (13 sequences)

SEQ ID NO: 1 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 2 - protein, 1243 aa, Homo sapiens
MLASPATETTVLMSQTEADLALRPPPPLGTAGQPRLGPPPRRARRFSGKAEPRPRSSRLSRRSSVDLGLLSSWSLPASPAPDPPDPPDSAGPGPARSPPPSSKEPPEGTWTEGAPVKAAEDSARPELPDSAVGPGSREPLRVPEAVALERRREQEEKEDMETQAVATSPDGRYLKFDIEIGRGSFKTVYRGLDTDTTVEVAWCELQTRKLSRAERQRFSEEVEMLKGLQHPNIVRFYDSWKSVLRGQVCIVLVTELMTSGTLKTYLRRFREMKPRVLQRWSRQILRGLHFLHSRVPPILHRDLKCDNVFITGPTGSVKIGDLGLATLKRASFAKSVIGTPEFMAPEMYEEKYDEAVDVYAFGMCMLEMATSEYPYSECQNAAQIYRKVTSGRKPNSFHKVKIPEVKEIIEGCIRTDKNERFTIQDLLAHAFFREERGVHVELAEEDDGEKPGLKLWLRMEDARRGGRPRDNQAIEFLFQLGRDAAEEVAQEMVALGLVCEADYQPVARAVRERVAAIQRKREKLRKARELEALPPEPGPPPATVPMAPGPPSVFPPEPEEPEADQHQPFLFRHASYSSTTSDCETDGYLSSSGFLDASDPALQPPGGVPSSLAESHLCLPSAFALSIPRSGPGSDFSPGDSYASDAASGLSDVGEGMGQMRRPPGRNLRRRPRSRLRVTSVSDQNDRVVECQLQTHNSKMVTFRFDLDGDSPEEIAAAMVYNEFILPSERDGFLRRIREIIQRVETLLKRDTGPMEAAEDTLSPQEEPAPLPALPVPLPDPSNEELQSSTSLEHRSWTAFSTSSSSPGTPLSPGNPFSPGTPISPGPIFPITSPPCHPSPSPFSPISSQVSSNPSPHPTSSPLPFSSSTPEFPVPLSQCPWSSLPTTSPPTFSPTCSQVTLSSPFFPPCPSTSSFPSTTAAPLLSLASAFSLAVMTVAQSLLSPSPGLLSQSPPAPPSPLPSLPLPPPVAPGGQESPSPHTAEVESEASPPPARPLPGEARLAPISEEGKPQLVGRFQVTSSKEPAEPLPLQPTSPTLSGSPKPSTPQLTSESSDTEDSAGGGPETREALAESDRAAEGLGAGVEEEGDDGKEPQVGGSPQPLSHPSPVWMNYSYSSLCLSSEESESSGEDEEFWAELQSLRQKHLSEVETLQTLQKKEIEDLYSRLGKQPPPGIVAPAAMLSSRQRRLSKGSFPTSRRNSLQRSEPPGPGIMRRNSLSGSSTGSQEQRASKGVTFAGDVGRM

SEQ ID NO: 3 - protein, 2297 aa, Homo sapiens
MDGDGGRRDVPGTLMEPGRGAGPAGMAEPRAKAARPGPQRFLRRSVVESDQEEPPGLEAAEAPGPQPPQPLQRRVLLLCKTRRLIAERARGRPAAPAPAALVAQPGAPGAPADAGPEPVGTQEPGPDPIAAAVETAPAPDGGPREEAAATVRKEDEGAAEAKPEPGRTRRDEPEEEEDDEDDLKAVATSLDGRFLKFDIELGRGSFKTVYKGLDTETWVEVAWCELQDRKLTKLERQRFKEEAEMLKGLQHPNIVRFYDFWESSAKGKRCIVLVTELMTSGTLKTYLKRFKVMKPKVLRSWCRQILKGLLFLHTRTPPIIHRDLKCDNIFITGPTGSVKIGDLGLATLKRASFAKSVIGTPEFMAPEMYEEHYDESVDVYAFGMCMLEMATSEYPYSECQNAAQIYRKVTCGIKPASFEKVHDPEIKEIIGECICKNKEERYEIKDLLSHAFFAEDTGVRVELAEEDHGRKSTIALRLWVEDPKKLKGKPKDNGAIEFTFDLEKETPDEVAQEMIESGFFHESDVKIVAKSIRDRVALIQWRRERIWPALQPKEQQDVGSPDKARGPPVPLQVQVTYHAQAGQPGPPEPEEPEADQHLLPPTLPTSATSLASDSTFDSGQGSTVYSDSQSSQQSVMLGSLADAAPSPAQCVCSPPVSEGPVLPQSLPSLGAYQQPTAAPGLPVGSVPAPACPPSLQQHFPDPAMSFAPVLPPPSTPMPTGPGQPAPPGQQPPPLAQPTPLPQVLAPQPVVPLQPVPPHLPPYLAPASQVGAPAQLKPLQMPQAPLQPLAQVPPQMPPIPVVPPITPLAGIDGLPPALPDLPTATVPPVPPPQYFSPAVILPSLAAPLPPASPALPLQAVKLPHPPGAPLAMPCRTIVPNAPATIPLLAVAPPGVAALSIHSAVAQLPGQPVYPAAFPQMAPTDVPPSPHHTVQNMRATPPQPALPPQPTLPPQPVLPPQPTLPPQPVLPPQPTRPPQPVLPPQPMLPPQPVLPPQPALPVRPEPLQPHLPEQAAPAATPGSQILLGHPAPYAVDVAAQVPTVPVPPAAVLSPPLPEVLLPAAPELLPQFPSSLATVSASVQSVPTQTATLLPPANPPLPGGPGIASPCPTVQLTVEPVQEEQASQDKPPGLPQSCESYGGSDVTSGKELSDSCEGAFGGGRLEGRAARKHHRRSTRARSRQERASRPRLTILNVCNTGDKMVECQLETHNHKMVTFKFDLDGDAPDEIATYMVEHDFILQAERETFIEQMKDVMDKAEDMLSEDTDADRGSDPGTSPPHLSTCGLGTGEESRQSQANAPVYQQNVLHTGKRWFIICPVAEHPAPEAPESSPPLPLSSLPPEASQGPCRGLTLPCLPWRRAACGAVFLSLFSAESAQSKQPPDSAPYKDQLSSKEQPSFLASQQLLSQAGPSNPPGAPPAPLAPSSPPVTALPQDGAAPATSTMPEPASGTASQAGGPGTPQGLTSELETSQPLAETHEAPLAVQPLVVGLAPCTPAPEAASTRDASAPREPLPPPAPEPSPHSGTPQPALGQPAPLLPAAVGAVSLATSQLPSPPLGPTVPPQPPSALESDGEGPPPRVGFVDSTIKSLDEKLRTLLYQEHVPTSSASAGTPVEVGDRDFTLEPLRGDQPRSEVCGGDLALPPVPKEAVSGRVQLPQPLVEKSELAPTRGAVMEQGTSSSMTAESSPRSMLGYDRDGRQVASDSHVVPSVPQDVPAFVRPARVEPTDRDGGEAGESSAEPPPSDMGTVGGQASHPQTLGARALGSPRKRPEQQDVSSPAKTVGRFSVVSTQDEWTLASPHSLRYSAPPDVYLDEAPSSPDVKLAVRRAQTASSIEVGVGEPVSSDSGDEGPRARPPVQKQASLPVSGSVAGDFVKKATAFLQRPSRAGSLGPETPSRVGMKVPTISVTSFHSQSSYISSDNDSELEDADIKKELQSLREKHLKEISELQSQQKQEIEALYRRLGKPLPPNVGFFHTAPPTGRRRKTSKSKLKAGKLLNPLVRQLKVVASSTGHLADSSRGPPAKDPAQASVGLTADSTGLSGKAVQTQQPCSVRASLSSDICSGLASDGGGARGQGWTVYHPTSERVTYKSSSKPRARFLSGPVSVSIWSALKRLCLGKEHSSRSSTSSLAPGPEPGPQPALHVQAQVNNSNNKKGTFTDDLHKLVDEWTSKTVGAAQLKPTLNQLKQTQKLQDMEAQAGWAAPGEARAMTAPRAGVGMPRLPPAPGPLSTTVIPGAAPTLSVPTPDGALGTARRNQVWFGLRVPPTACCGHSTQPRGGQRVGSKTASFAASDPVRS

SEQ ID NO: 4 - protein, 1800 aa, Homo sapiens
MATDSGDPASTEDSEKPDGISFENRVPQVAATLTVEARLKEKNSTFSASGETVERKRFFRKSVEMTEDDKVAESSPKDERIKAAMNIPRVDKLPSNVLRGGQEVKYEQCSKSTSEISKDCFKEKNEKEMEEEAEMKAVATSPSGRFLKFDIELGRGAFKTVYKGLDTETWVEVAWCELQDRKLTKAEQQRFKEEAEMLKGLQHPNIVRFYDSWESILKGKKCIVLVTELMTSGTLKTYLKRFKVMKPKVLRSWCRQILKGLQFLHTRTPPIIHRDLKCDNIFITGPTGSVKIGDLGLATLMRTSFAKSVIGTPEFMAPEMYEEHYDESVDVYAFGMCMLEMATSEYPYSECQNAAQIYRKVTSGIKPASFNKVTDPEVKEIIEGCIRQNKSERLSIRDLLNHAFFAEDTGLRVELAEEDDCSNSSLALRLWVEDPKKLKGKHKDNEAIEFSFNLETDTPEEVAYEMVKSGFFHESDSKAVAKSIRDRVTPIKKTREKKPAGCLEERRDSQCKSMGNVFPQPQNTTLPLAPAQQTGAECEETEVDQHVRQQLLQRKPQQHCSSVTGDNLSEAGAASVIHSDTSSQPSVAYSSNQTMGSQMVSNIPQAEVNVPGQIYSSQQLVGHYQQVSGLQKHSKLTQPQILPLVQGQSTVLPVHVLGPTVVSQPQVSPLTVQKVPQIKPVSQPVGAEQQAALLKPDLVRSLNQDVATTKENVSSPDNPSGNGKQDRIKQRRASCPRPEKGTKFQLTVLQVSTSGDNMVECQLETHNNKMVTFKFDVDGDAPEDIADYMVEDNFVLESEKEKFVEELRAIVGQAQEILHVHFATERATGVDSITVDSNSSQTGSSEQVQINSTSTQTSNESAPQSSPVGRWRFCINQTIRNRETQSPPSLQHSMSAVPGRHPLPSPKNTSNKEISRDTLLTIENNPCHRALFTSKSEHKDVVDGKISECASVETKQPAILYQVEDNRQIMAPVTNSSSYSTTSVRAVPAECEGLTKQASIFIPVYPCHQTASQADALMSHPGESTQTSGNSLTTLAFDQKPQTLSVQQPAMDAEFISQEGETTVNTEASSPKTVIPTQTPGLEPTTLQPTTVLESDGERPPKLEFADNRIKTLDEKLRNLLYQEHSISSIYPESQKDTQSIDSPFSSSAEDTLSCPVTEVIAISHCGIKDSPVQSPNFQQTGSKLLSNVAASQPANISVFKRDLNVITSVPSELCLHEMSSDASLPGDPEAYPAAVSSGGAIHLQTGGGYFGLSFTCPSLKNPISKKSWTRKLKSWAYRLRQSTSFFKRSKVRQVETEEMRSAIAPDPIPLTRESTADTRALNRCKAMSGSFQRGRFQVITIPQQQSAKMTSFGIEHISVFSETNHSSEEAFIKTAKSQLVEIEPATQNPKTSFSYEKLQALQETCKENKGVPKQGDNFLSFSAACETDVSSVTPEKEFEETSATGSSMQSGSELLLKEREILTAGKQPSSDSEFSASLAGSGKSVAKTGPESNQCLPHHEEQAYAQTQSSLFYSPSSPMSSDDESEIEDEDLKVELQRLREKHIQEVVNLQTQQNKELQELYERLRSIKDSKTQSTEIPLPPASPRRPRSFKSKLRSRPQSLTHVDNGIVATGKSCLINELENPLCVESNAASCQQSPASKKGMFTDDLHKLVDDWTKEAVGNSLIKPSLNQLKQSQHKLETENWNKVSENTPSTMGYTSTWISSLSQIRGAVPTSLPQGLSLPSFPGPLSSYGMPHVCQYNAVAGAGYPVQWVGISGTTQQSVVIPAQSGGPFQPGMNMQAFPTSSVQNPATIPPGPK

SEQ ID NO: 5 - protein, 1243 aa, Homo sapiens; identical to SEQ ID NO: 2

SEQ ID NO: 6 - protein, 545 aa, Homo sapiens
MAEPSGSPVHVQLPQQAAPVTAAAAAAPAAATAAPAPAAPAAPAPAPAPAAQAVGWPICRDAYELQEVIGSGATAVVQAALCKPRQERVAIKRINLEKCQTSMDELLKEIQAMSQCSHPNVVTYYTSFVVKDELWLVMKLLSGGSMLDIIKYIVNRGEHKNGVLEEAIIATILKEVLEGLDYLHRNGQIHRDLKAGNILLGEDGSVQIADFGVSAFLATGGDVTRNKVRKTFVGTPCWMAPEVMEQVRGYDFKADMWSFGITAIELATGAAPYHKYPPMKVLMLTLQNDPPTLETGVEDKEMMKKYGKSFRKLLSLCLQKDPSKRPTAAELLKCKFFQKAKNREYLIEKLLTRTPDIAQRAKKVRRVPGSSGHLHKTEDGDWEWSDDEMDEKSEEGKAAFSQEKSRRVKEENPEIAVSASTIPEQIQSLSVHDSQGPPNANEDYREASSCAVNLVLRLRNSRKELNDIRFEFTPGRDTADGVSQELFSAGLVDGHDVVIVAANLQKIVDDPKALKTLTFKLASGCDGSEIPDEVKLIGFAQLSVS

SEQ ID NO: 7 - protein, 527 aa, Homo sapiens
MSEDSSALPWSINRDDYELQEVIGSGATAVVQAAYCAPKKEKVAIKRINLEKCQTSMDELLKEIQAMSQCHHPNIVSYYTSFVVKDELWLVMKLLSGGSVLDIIKHIVAKGEHKSGVLDESTIATILREVLEGLEYLHKNGQIHRDVKAGNILLGEDGSVQIADFGVSAFLATGGDITRNKVRKTFVGTPCWMAPEVMEQVRGYDFKADIWSFGITAIELATGAAPYHKYPPMKVLMLTLQNDPPSLETGVQDKEMLKKYGKSFRKMISLCLQKDPEKRPTAAELLRHKFFQKAKNKEFLQEKTLQRAPTISERAKKVRRVPGSSGRLHKTEDGGWEWSDDEFDEESEEGKAAISQLRSPRVKESISNSELFPTTDPVGTLLQVPEQISAHLPQPAGQIATQPTQVSLPPTAEPAKTAQALSSGSGSQETKIPISLVLRLRNSKKELNDIRFEFTPGRDTAEGVSQELISAGLVDGRDLVIVAANLQKIVEEPQSNRSVTFKLASGVEGSDIPDDGKLIGFAQLSIS

SEQ ID NO: 8 - DNA, 21 bp, synthetic construct
  source 1..21: mol_type = other DNA
gccgtcagatccttaaaggtc

SEQ ID NO: 9 - DNA, 19 bp, synthetic construct
  source 1..19: mol_type = other DNA
ccagtagggccggtgataa

SEQ ID NO: 10 - DNA, 21 bp, synthetic construct
  source 1..21: mol_type = other DNA
catacctgaagcggttcaagg

SEQ ID NO: 11 - DNA, 22 bp, synthetic construct
  source 1..22: mol_type = other DNA
cttttggcaaatgacgctcttt

SEQ ID NO: 12 - DNA, 20 bp, synthetic construct
  source 1..20: mol_type = other DNA
tgcgtaaactggatgacacc

SEQ ID NO: 13 - DNA, 21 bp, synthetic construct
  source 1..21: mol_type = other DNA
cctgctttggtatggagagtc